(19) United States
(12) Patent Application Publication
(10) Pub. No.: US 2017/0165375 A1
(43) Pub. Date: Jun. 15, 2017

(54) ANTIBIOTIC PROTOCELLS AND RELATED PHARMACEUTICAL FORMULATIONS AND METHODS OF TREATMENT

(71) Applicants: STC. UNM, ALBUQUERQUE, NM (US); SANDIA CORPORATION, ALBUQUERQUE, NM (US)

(72) Inventors: Carlee Erin Ashley, Albuquerque, NM (US); Eric C. Carnes, Albuquerque, NM (US); Terry Wu, Albuquerque, NM (US); Linda A. Felton, Albuquerque, NM (US); Darryl Y. Sasaki, Livermore, CA (US)

(21) Appl. No.: 14/781,817

(22) PCT Filed: Apr. 2, 2014
(86) PCT No.: PCT/US2014/032702

§ 371 Date: Nov. 9, 2015

Related U.S. Application Data
(60) Provisional application No. 61/807,679, filed on Apr. 2, 2013.

(51) Int. Cl. A61K 9/51; A61K 9/50; A61K 31/5383; A61K 31/65; A61K 31/546; A61K 31/635; A61K 31/505; A61K 9/00; A61K 9/127; A61K 31/7036

(52) U.S. Cl. CPC A61K 47/48823; A61K 9/1271; A61K 9/5021; A61K 47/48015; A61K 47/48861; A61K 47/48838; A61K 47/48369; A61K 31/5383; A61K 47/48238; A61K 31/65; A61K 31/7036; A61K 31/546; A61K 31/635; A61K 31/505; A61K 9/0053

(57) ABSTRACT

The invention provides novel antibiotic protocells comprising mesoporous nanoparticles encapsulated within a lipid bi- or multilayer. The nanoparticles have pore sizes and surface chemistries that enable facile adsorption and intracellular presentation of antibiotics which are effective in the treatment of a wide variety of bacterial infections, including F. tularensis, B. pseudomallei and P. aeruginosa-related infections. Related pharmaceutical compositions and methods of treatment are also provided.

### CROSS REFERENCE TO RELATED APPLICATIONS

This application claims priority from U.S. Provisional Patent Application No. 61/807,679, entitled “Protocell Nanoparticles for Sustained Delivery of Antibiotics”, filed Apr. 2, 2013. The complete contents of this provisional application are hereby incorporated by reference in their entirety.

### STATEMENT REGARDING FEDERAL FUNDING

This invention was developed under Contract DE-AC04-94AL85000 between Sandia Corporation and the U.S. Department of Energy. Accordingly, the United States has certain rights in this invention.

### FIELD OF THE INVENTION

The present invention relates to antibiotic protocells that are useful in the treatment of infections caused by a wide variety of bacteria, including infections caused by Francisella tularensis, Burkholderia pseudomallei and Pseudomonas aeruginosa, among others.

### BACKGROUND OF THE INVENTION

Francisella tularensis is the etiological agent of tularemia, a serious and occasionally fatal disease of humans and animals. In humans, ulceroglandular tularemia is the most common form of the disease and is usually a consequence of a bite from an arthropod vector which has previously fed on an infected animal. The pneumonic form of the disease occurs rarely but is the likely form of the disease should this bacterium be used as a bioterrorism agent. Ellis, et. al., Clin. Microbiol. Rev. October 2002 vol. 15 no. 4 631-646.

Burkholderia pseudomallei (previously called Pseudomonas pseudomallei) is the causative agent of melioidosis, a serious disease of man and animals that occurs primarily in S.E. Asia, N. Australia and other tropical areas. B. pseudomallei is an environmental Gram-negative saprophyte present in wet soil and rice paddies in endemic areas. The highest documented infection rate is in northeastern Thailand, where melioidosis accounts for 20% of all community-acquired septicaemias. Disease occurs after bacterial contamination of breaks in the skin or by inhalation after contact with water or soil. There is no licensed vaccine against meliodosis, and the bacterium is resistant to many antibiotics.

Bacteria of the genus Pseudomonas thrive in hospitals and are the second most common cause of hospital infections. As explained in United States Patent Application Document No. 20140065183, “persistent infections resulting from Pseudomonas aeruginosa are the major cause of morbidity and mortality in cystic fibrosis (CF) patients, and in those with burns, neutropenia or with otherwise compromised immunity. P. aeruginosa is notoriously difficult to eradicate even with long-term antibiotic therapy and there is evidence that the ability of the organism to form biofilms contributes to this resistance.”

In order to effectively kill intracellular F. tularensis, B. pseudomallei and P. aeruginosa, and the other bacteria described herein, nanoparticle delivery vehicles must release antibiotics directly into the cytosol of host cells. This not only increases the concentration of drug in the vicinity of the pathogen, but is also important since many classes of antibiotics, including β-lactams, lincosamides, and fluoroquinolones, show poor penetration into or rapid efflux from mammalian and/or microbial cells. A number of factors govern cellular uptake and processing of nanoparticles, including their size, shape, surface charge, and degree of hydrophobicity. Additionally, a variety of molecules, including peptides, proteins, aptamers, and antibodies, can be employed to trigger active uptake by a plethora of specific cells.

The need exists for therapeutic protocells which can be made by commercially practicable processes, which target delivery of antibiotics to a host cell's cytosol and which are effective in the treatment of bacterial infections, including F. tularensis, B. pseudomallei and P. aeruginosa-related infections, among others. There is a particular need for an enhanced therapeutically-effective, nanoparticulate-based, oral formulation that can be used in the treatment of respiratory tularemia and other infections of the lung.

### SUMMARY OF THE INVENTION

We have developed novel antibiotic protocells comprising mesoporous nanoparticles encapsulated within a lipid bi- or multilayer. The nanoparticles have pore sizes and surface chemistries that enable facile adsorption and intracellular presentation of antibiotics which are effective in the treatment of a wide variety of bacterial infections, including F. tularensis, B. pseudomallei and P. aeruginosa-related infections.

In one embodiment, the invention provides a novel protocell which targets one or more bacterially-infected host cells and which comprises:

(a) mesoporous silica nanoparticles (MSNPs) comprising about 10% to about 70% by weight of one or more antibiotics and having a pore size of approximately 1 nm to approximately 75 nm (a distinction from IUPAC defined mesopores), a surface area of approximately 75 m²/g to approximately 1,500 m²/g and a hydrodynamic diameter of approximately 50 nm to approximately 50 μm; and
(b) a lipid bi- or multilayer which encapsulates the nanoparticle and which preferably comprises a mixture of pegylated and non-pegylated lipids; and optionally, (1) an optionally-thiolated PEG and/or (2) at least one targeting ligand which is conjugated to the outer surface of the lipid bi- or multilayer and which is specific against one or more receptors of a bacterially-infected host cell. In preferred aspects, the PEGylated lipid bi- or multilayer comprises about 0% to about 25%, preferably about 0.001% to about 20-25% by weight (of the total amount of lipid) of a pegylated lipid and about 75% to about 100% or about 75% to about 99.999% by weight of a non-pegylated lipid. In certain embodiments, the PEG is crosslinked with cholesterol and/or bonded to another lipid component in which case the PEGylated cholesterol component may be the only PEGylated component in the lipid bilayer/multilayer (at a level of about 0.001% to about 20-25%, often about 0.1% to about 15%, about 1% to about 10%), but may be included with other PEGylated lipids in certain aspects of the invention.

Preferably, the MSNPs are made by an aerosol-assisted evaporation-induced self-assembly (EISA) process in which charge and/or hydrophobicity of the MSNPs are varied by addition of one or more aminosilanes and/or trimethylsilyl group capping agents depending upon the charge and/or hydrophobicity of the one or more antibiotics, and wherein the maximum concentration of antibiotic loaded within the nanoparticle's pore network is approximately equal to the antibiotic's maximum solubility in its ideal solvent. We have discovered that a protocell's antibiotic release profile is dependent on the extent of MSNP silica framework condensation during EISA.

In certain embodiments, the mesoporous silica nanoparticle is aminated (or has another functional group such as a glycidoxy group and is conjugated to the lipid bi- or multilayer by cholesterol-containing tether molecules which form an amine-coupled linkage or directly to the silica nanoparticle through the glycidoxy group or other silyl group) between the mesoporous silica nanoparticle and cholesterol (often through a urethane, amide, ether or other linkage) which spaces the nanoparticle and the lipid bi- or multilayer through cholesterol. These tether molecules provide additional spacing between the lipid bilayer/multilayer and the silica nanoparticle. Additionally, the cholesterol-containing tether molecules can contain a PEG group which is covalently bound to the hydroxyl group of cholesterol (through a linking group) and the PEG group to the amine-group of the amine-modified nanoparticle through a linking group or alternatively through a glycidoxy or a silyl group on the nanoparticle. As discussed above, cholesterol may be PEGylated (without being bound to the nanoparticle) to provide a further or alternative PEGylated lipid compound to lipid bi- and multi-layers pursuant to the present invention.

EISA provides silica nanoparticles which are mesoporous, which can be stably loaded with high concentrations of various protein antigens and antibiotics, and which may be engineered for burst or sustained release profiles by modifying the condensation (density and number of pores) of the nanoparticle to influence the release characteristics of the nanoparticle. A great condensation (greater density/fewer pores) will generally increase the release times, whereas a lower/lesser condensation/density/more pores will reduce release times. Significantly, EISA enables hydrodynamic size to be varied from 20-nm to greater than 10-μm and enables pore walls to be modified with a wide range of functional moieties that facilitate high capacity loading of physicochemically disparate diagnostic and/or therapeutic molecules. Importantly, EISA also produces MSNPs that can be easily dispersed in a variety of aqueous and organic solvents without any appreciable aggregation, which enables us to load drugs that have high and low solubility in water. Our MSNPs are also easily encapsulated within anionic, cationic, and electrically-neutral SLBs via simple liposome fusion.

MSNPs formed by EISA have loading capacities of 20-55 wt % for various individual antibiotics and about 10-15 wt % for individual antibiotics in three-drug-cocktails. Significantly, these capacities are 10-fold higher than other MSNP-based delivery vehicles and 100-1,000-fold higher than similarly-sized liposomes and polymeric nanoparticles.

Our novel protocells can be used to treat a wide variety of bacterial infections including, but are not limited to, infections caused by bacteria selected from the group consisting of F. tularensis, B. pseudomallei, Mycobacterium, staphylococcus, streptococcaceae, neisseriaaceae, cocci, enterobacteriaceae, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae and P. aeruginosa

In a preferred embodiment, the lipid bi- or multilayer comprises:

(a) at least one zwitterionic lipid selected from the group consisting of 1, 2-dioleoyl-sn-glycero-3-phosphocholine (DOPC), 1, 2-dioleoyl-sn-glycero-3-phosphoethanolamine (DOPE) (which is optionally pegylated) and 1,2-distearoyl-sn-glycero-3-phosphocholine (DSPC);
(b) one or more targeting ligands selected from the group consisting of RGD (Arg-Gly-Asp), Fcγ (synthesized with a C-terminal cysteine residue), human complement C3, ephrin B2 and SP94;
(c) an endo/lyso/phagosomolytic peptide (e.g. H5WYG (synthesized with a C-terminal cysteine residue));
(d) optionally, the self signal CD47 and/or a polymerizable lipid; and
(e) optionally, one or more nucleic acid sequences that are selected from the group consisting of small interfering RNA, small hairpin RNA, microRNA and spherical nucleic acids (SNAs), the nucleic acid sequences being complementary to one or more of a β-lactamase gene, a single-gene determinant of antibiotic resistance, a gene that contributes to virulence and a RNA polymerase or gyrase.

As explained hereinafter, an endo/lyso/phagosomolytic peptide such as H5WYG can be conjugated to diacylphosphatidylethanolamine (PE) moieties on the surface of the lipid bi- or multilayer by an amine-to-sulfhydryl crosslinker with a PEG spacer, and a targeting ligand such as RGD (Arg-Gly-Asp) can be conjugated to the surface of the lipid bi- or multilayer by an acid labile crosslinker.

In certain embodiments:

(1) the host cells are infected by F. tularensis, B. pseudomallei or P. aeruginosa and the nanoparticles contain one or more nucleic acid sequences that are complementary to a gene sequence expressed by F. tularensis, B. pseudomallei or P. aeruginosa; and
(2) and the lipid bi- or multilayer comprises one or more targeting ligands selected from the group consisting of Fcγ from human IgG (which binds to Fcγ receptors on macrophages and dendritic cells), human complement C3 (which binds to CR1 on macrophages and dendritic cells), ephrin B2 (which binds to EphB4 receptors on alveolar type II epithelial cells), and the SP94 peptide (which binds to unknown receptor(s) on hepatocyte-derived cells).

In certain preferred embodiments, the lipid bi- or multilayers comprise:

(1) a moderate density of PEG (which has been shown to enhance mucosal penetration in the intestine and reduce RES clearance of nanoparticles); and
(2) a high density of RGD (Arg-Gly-Asp) peptide (which is known to trigger transcytosis across intestinal M cells).

Our invention also includes the use of our novel protocells in pharmaceutical compositions and in the treatment of infections caused by the above-described bacteria.

In a preferred embodiment, the invention provides an oral formulation which is useful in the treatment of respiratory tularemia and which comprises a plurality of protocells comprised of mesoporous, negatively-charged silica nanoparticle cores that are loaded with one or more antibiotics (i) that are selected from the group consisting of a tetracycline (preferably doxycycline), a fluoroquinolone (preferably ciprofloxacin) and Streptomycin or gentamicin, and that are (ii) encapsulated within supported lipid bi- or multilayers comprising from about 35-55 wt % 1,2-dioleoyl-sn-glycero-3-phosphocholine (DOPC), 5-15 wt % of 1,2-dioleoyl-sn-glycero-3-phosphoethanolamine (DOPE), 30-40 wt % of cholesterol, and 10-20 wt % of PEG. In certain aspects, the PEG is either bonded to DOPE and/or crosslinked with a portion of the cholesterol.

As explained in further detail herein, protocells of the invention are highly flexible and modular. High concentrations of physicochemically-disparate molecules can be loaded into the protocells and their antibiotic release rates can be optimized without altering the protocell's size, size distribution, stability, or synthesis strategy. Properties of the supported lipid bilayer and mesoporous silica nanoparticle core can also be modulated independently, thereby optimizing properties as surface charge, colloidal stability, and targeting specificity independently from overall size, type of cargo(s), loading capacity, and release rate.

These and other aspects of the invention are described further in the Detailed Description of the Invention.

### BRIEF DESCRIPTION OF THE FIGURES

FIG. 1. Protocells have a high capacity for physicochemically disparate therapeutic agents, tailorable release rates, and exquisite targeting specificities, all of which enable levofloxacin-loaded, Fcγ-targeted protocells to effectively kill intracellular Ft in a cell-specific fashion. (A) The loading capacities of MSNPs with 2.5-nm pores for several physicochemically disparate antibiotics. (B) Time-dependent release of levofloxacin from MSNPs with a low or high degree of silica (SiO2) framework condensation upon incubation in a simulated body fluid (10% serum, pH 7.4) at 37° C. (C) Mean fluorescence intensities of THP-1, A549, and HepG2 after incubation with DOPC protocells labeled with pHrodo Red and modified with 5 wt % of human Fcγ, 5 wt % of human complement C3, 30 wt % of mannosylated cholesterol, 5 wt % of human ephrin B2, or 5 wt % of SP94. (D) Number of colony-forming units (CFUs) of Ft LVS that remain upon treatment of LVS-infected THP-1 or A549 cells with empty protocells, free levofloxacin (Levo), or levofloxacin loaded in DOPC protocells or DPPC liposomes modified with 5 wt % of Fcγ and H5WYG. Error bars=mean±std. dev. for n=3.

FIG. 2. FIG. 2 illustrates a protocell of the invention.

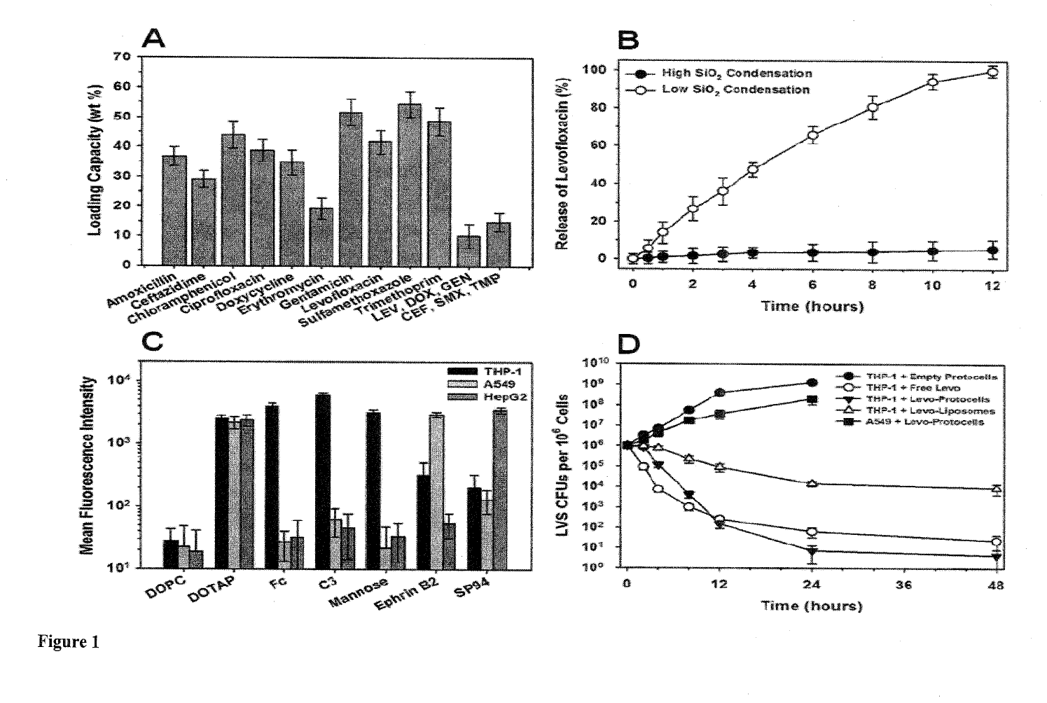

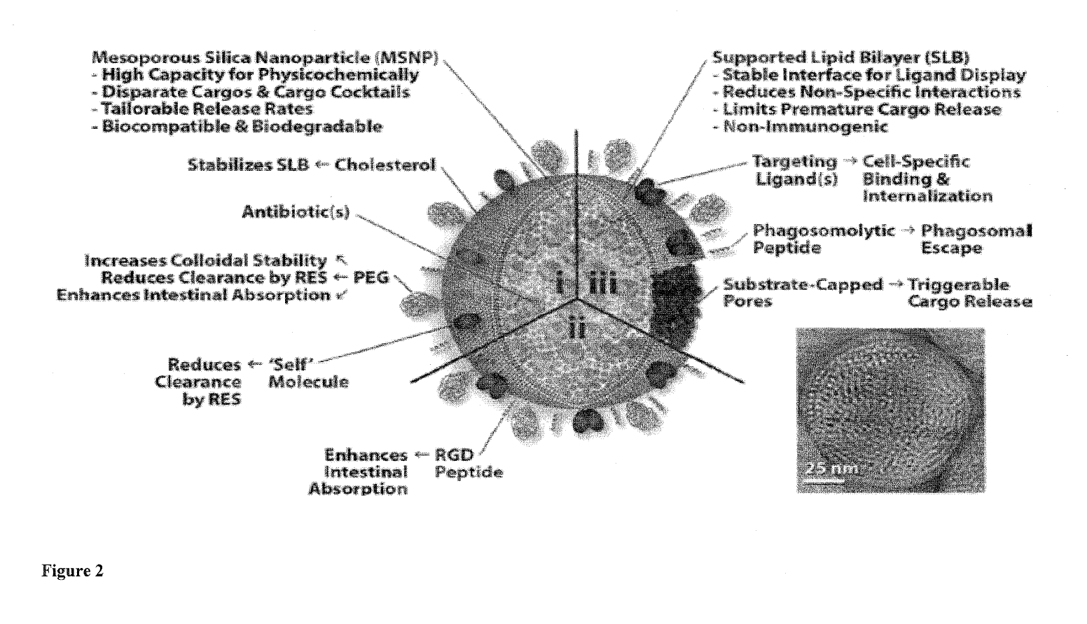

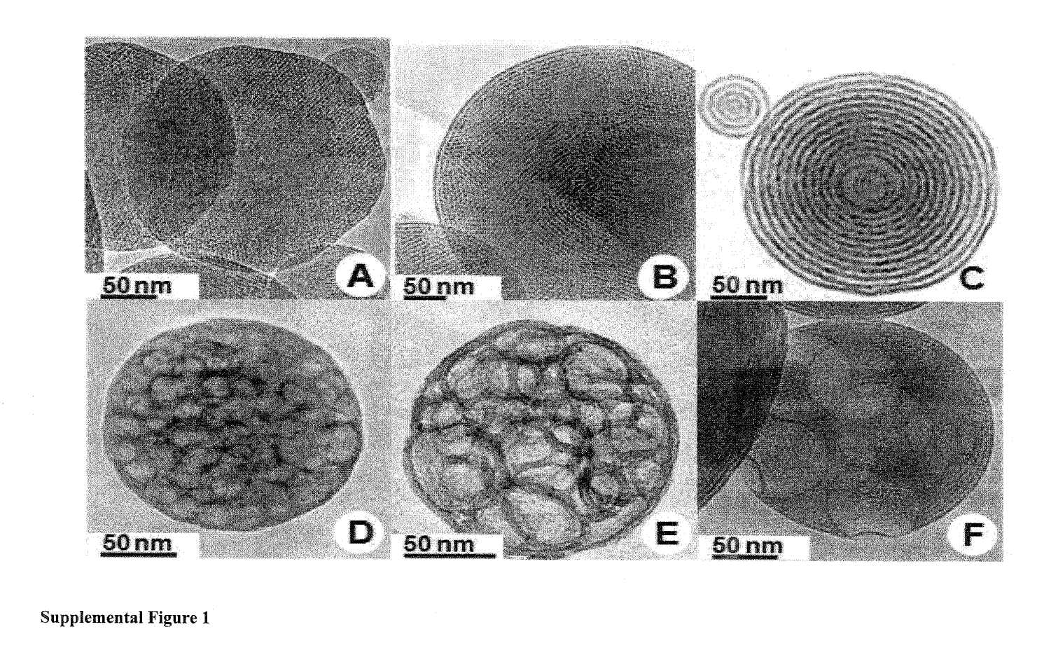

Supplementary FIG. 1. Gallery of mesoporous silica nanoparticles prepared by aerosol-assisted EISA with hexagonal (A), cubic (B), lamellar (C), and cellular (D-E) pore geometries. (F) shows dual-templated particles with interconnected 2-nm and 60-nm pores. See Lu, Brinker, et al. Nature (1999) for further details.

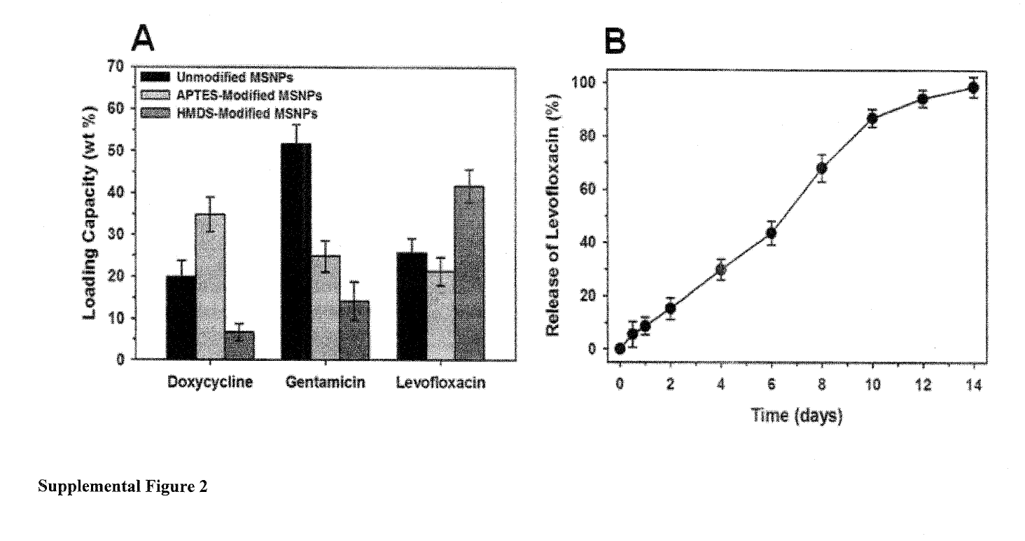

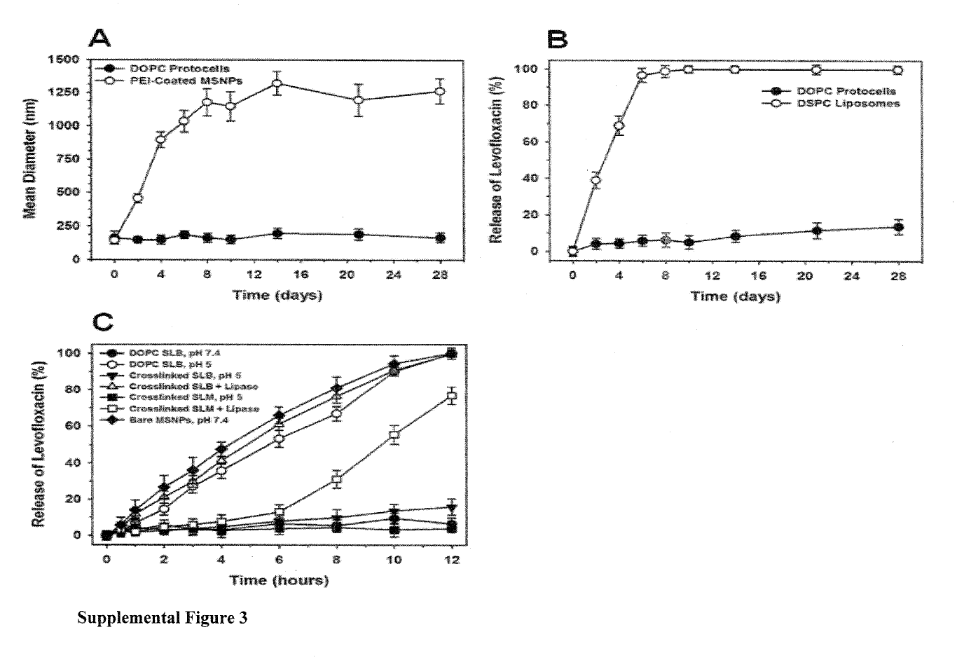

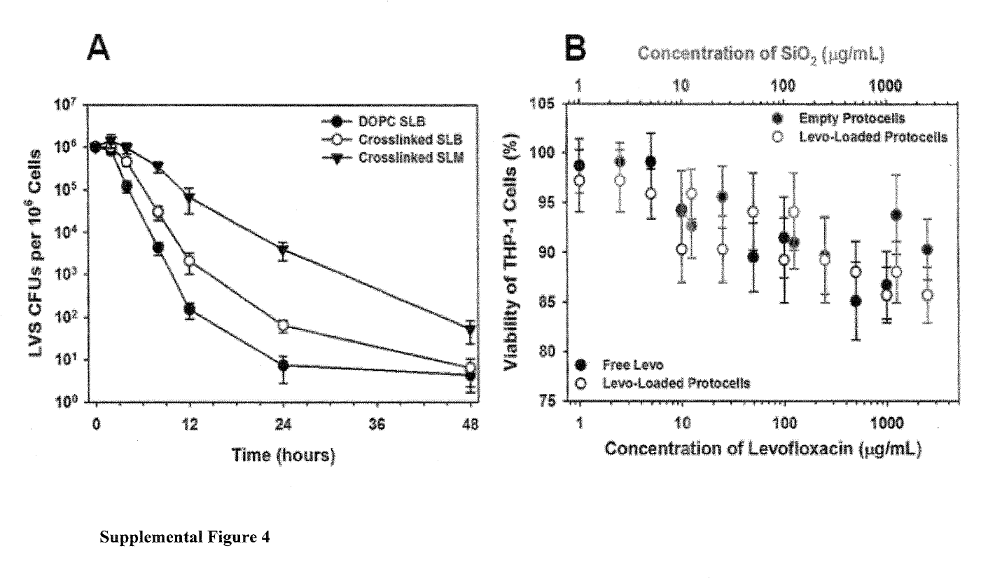

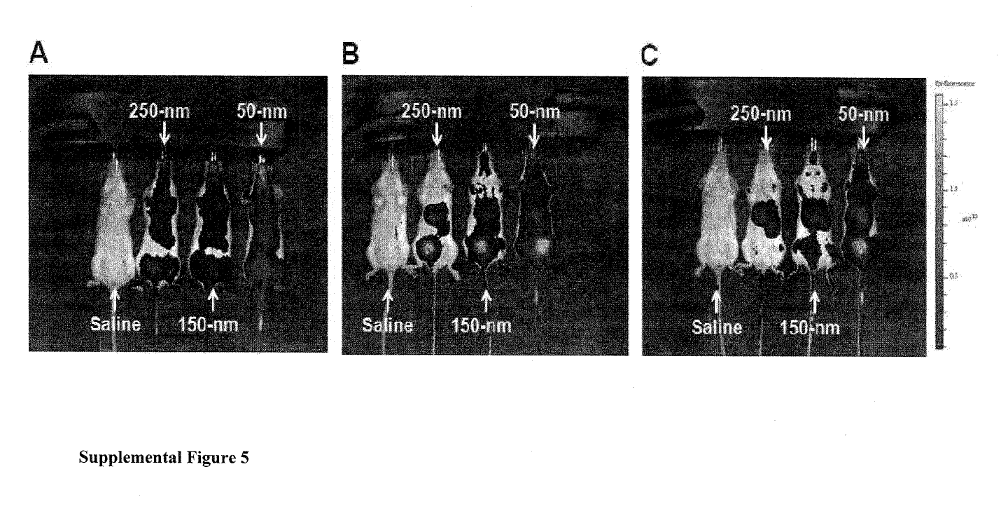

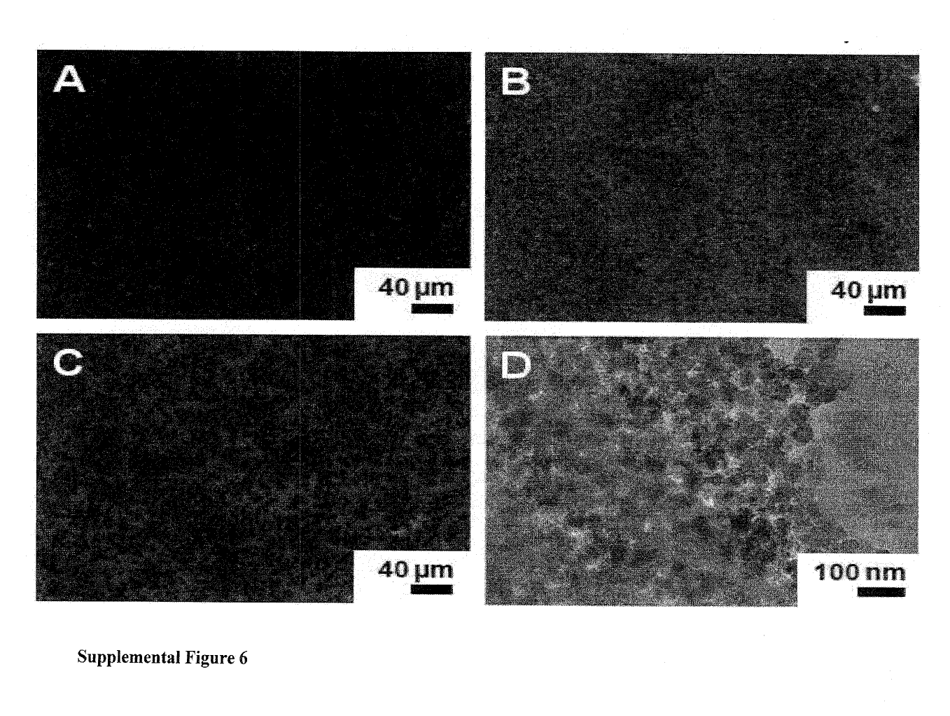

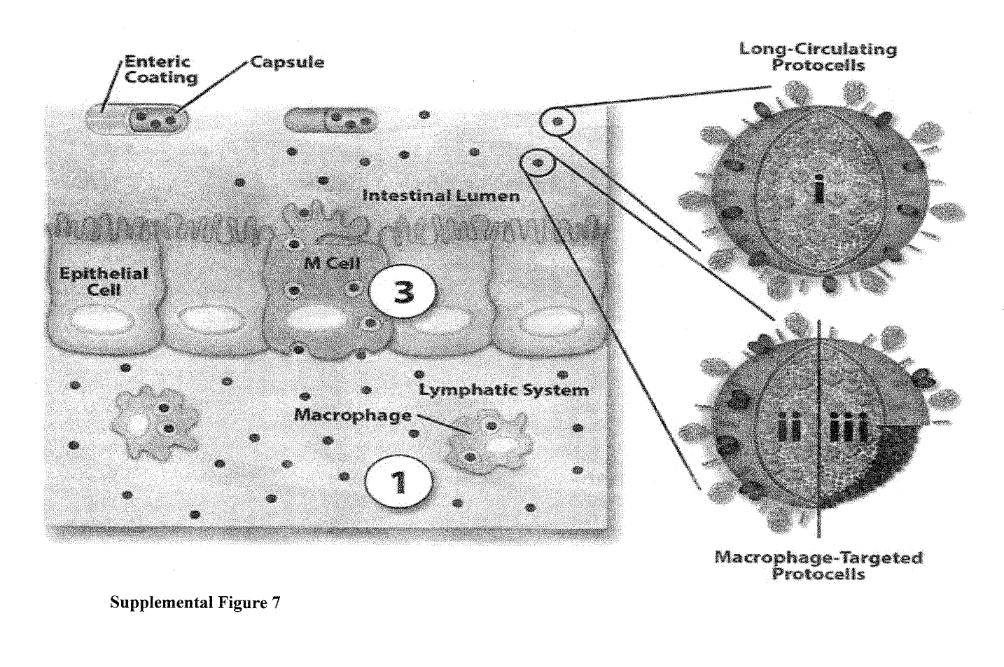

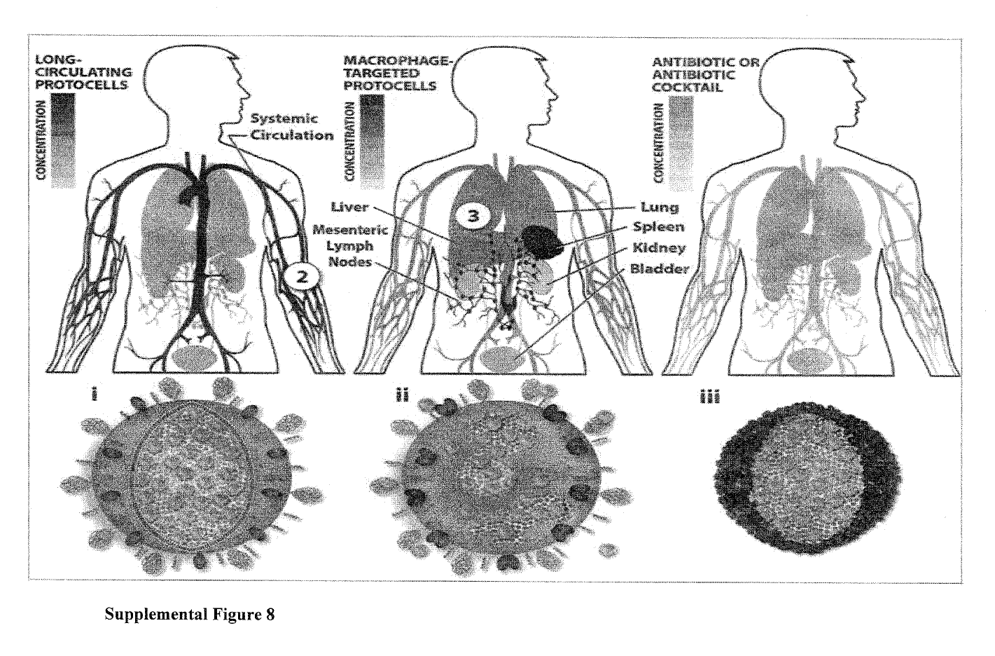

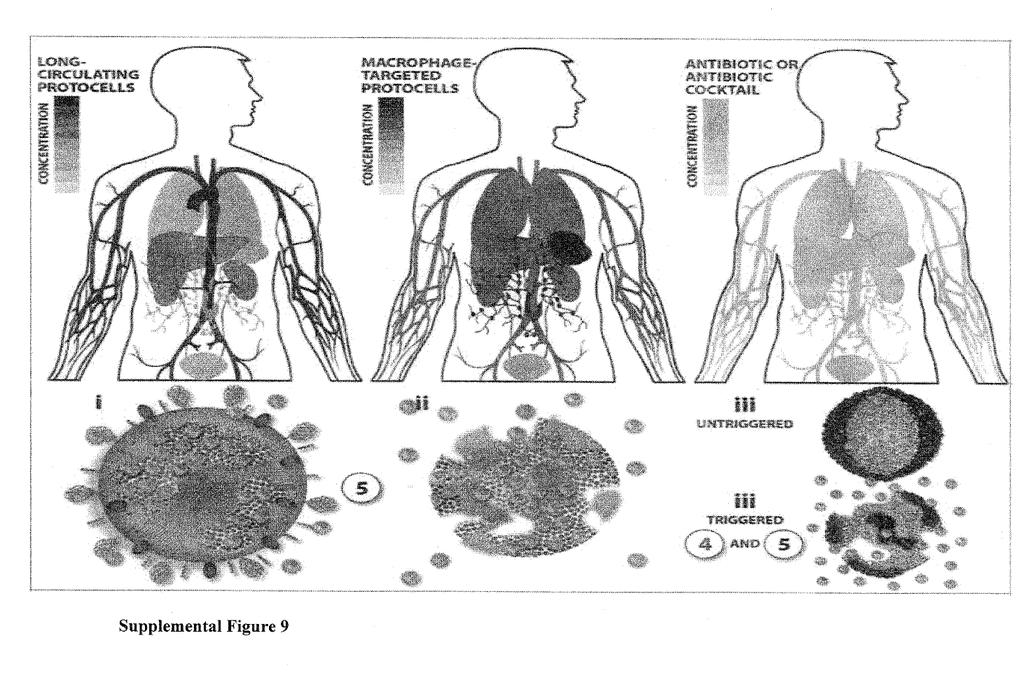

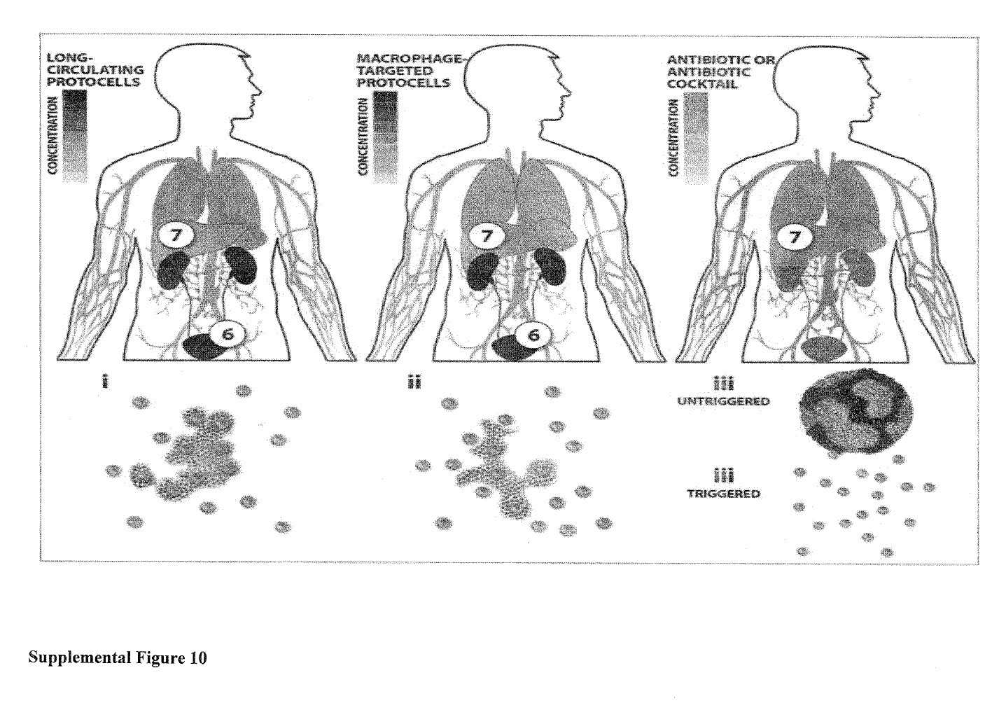

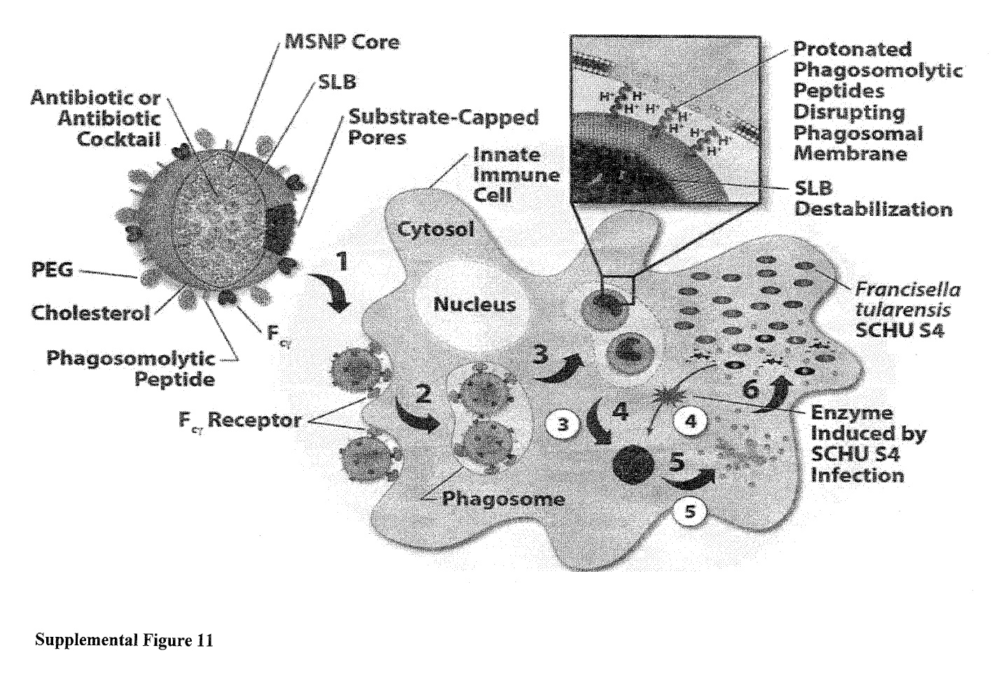

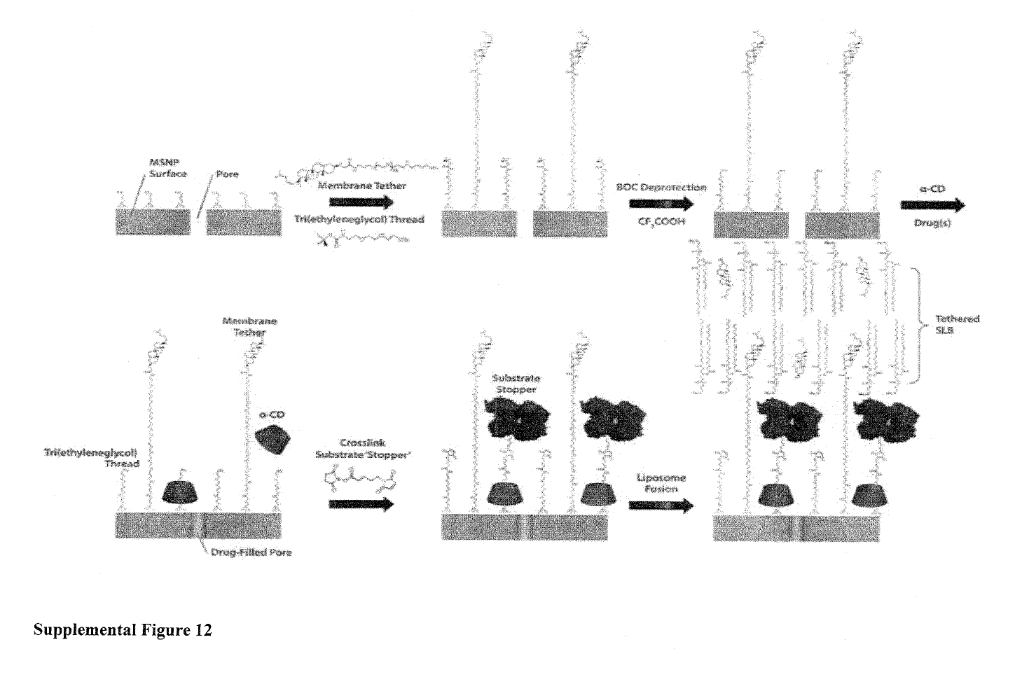

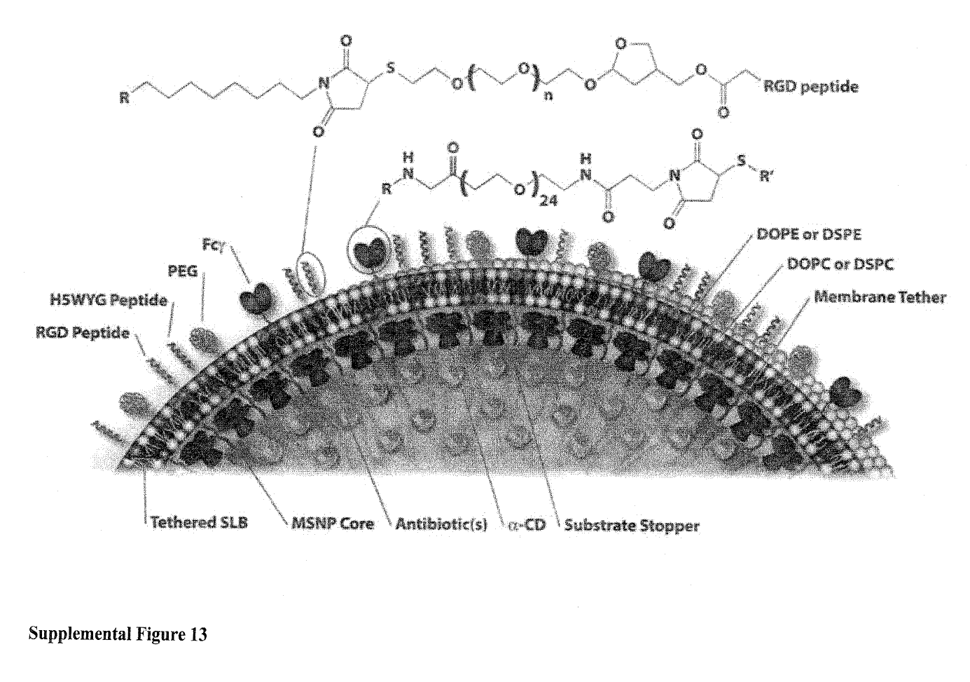

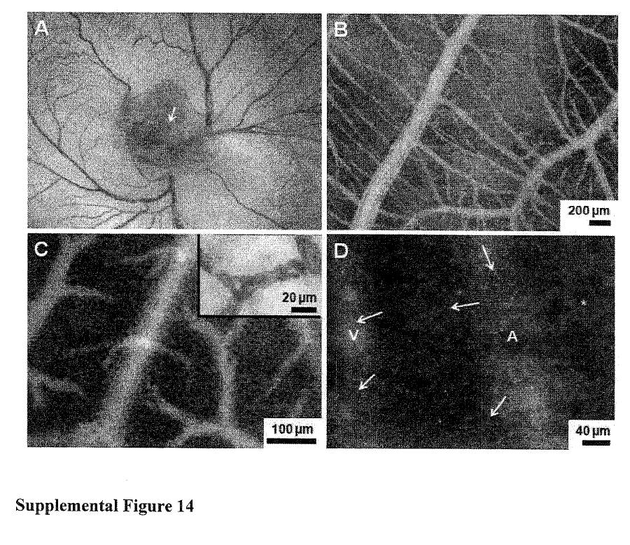

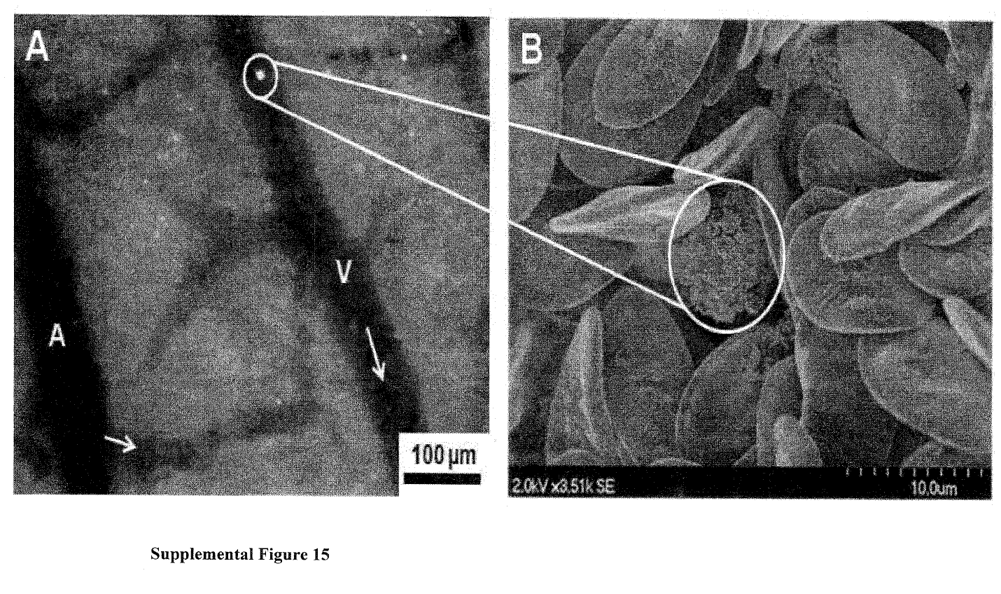

Supplementary FIG. 2. MSNPs generated via aerosol-assisted EISA have a high capacity for physicochemically disparate antibiotics, the release rates of which can be tailored by altering the degree of condensation of the MSNP framework. (A) The loading capacities of MSNPs for acidic (doxycycline, pKa=4.7), basic (gentamicin, pKa=13.2), and hydrophobic (levofloxacin, log P=2.1) drugs can be enhanced by altering the charge or degree of hydrophobicity of the MSNP framework. MSNPs are naturally negatively-charged (ζ=−20 mV in 0.5×PBS, pH 7.4) but were modified with (3-aminopropyl)triethoxysilane (APTES) to make pores positively-charged (ζ=+25 mV in 0.5×PBS, pH 7.4) and with hexamethyldisilazane (HMDS) to make pores more hydrophobic. (B) The percentage of levofloxacin released from MSNPs with a high degree of silica framework condensation upon incubation in a simulated body fluid (10% serum, pH 7.4) at 37° C. for the indicated periods of time. Data represent the mean±std. dev. for n=3. Unpublished data.

Supplementary FIG. 3. Encapsulation of levofloxacin-loaded MSNPs in a PEGylated SLB enables long-term colloidal stability and drug retention in simulated body fluids, and SLB stability can be modulated to control release of levofloxacin. (A) The mean hydrodynamic diameters, as determined by DLS, of DOPC protocells and PEI-coated MSNPs upon incubation in a simulated body fluid (10% FBS, pH 7.4) at 37° C. for the indicated periods of time. (B) The percentage of levofloxacin released from DOPC protocells and DSPC liposomes upon incubation in a simulated body fluid (10% serum, pH 7.4) at 37° C. for the indicated periods of time. (C) The percentage of levofloxacin released from protocells with SLBs composed of DOPC (‘DOPC SLB’), SLBs composed of 70 wt % DOPC and 30 wt % of photopolymerized 16:0-23:2 Diyene PC (‘Crosslinked SLB’), and SLMs composed of 70% DOPC with 30 wt % 18:1 MPB PE (‘Crosslinked SLM’) upon incubation in a simulated body fluid (10% serum, pH 7.4), 50 mM sodium citrate at pH 5.0, or 1×PBS with 500 ng/mL of phospholipase A at 37° C. for the indicated periods of time. The levofloxacin release profile for MSNPs without a SLB is included for comparison. MSNPs with a low degree of silica condensation were used in all experiments. Protocell SLBs were composed of DOPC with 30 wt % of cholesterol and 10 wt % of PEG unless otherwise noted. Liposomes in (B) were composed of DSPC with 30 wt % cholesterol and 10 wt % of PEG. Data represent the mean±std. dev. for n=3. Unpublished data.

Supplementary FIG. 4. Modification of the SLB with targeting ligands promotes efficient, cell-specific internalization of levofloxacin-loaded protocells, which enables highly efficacious killing of intracellular F. tularensis without substantially impacting host cell viability. (A) The number colony-forming units (CFUs) of Ft LVS that remain upon incubation of LVS-infected THP-1 cells and Fcγ-targeted protocells with SLBs composed of DOPC, DOPC with 50 wt % of 16:0-23:2 Diyene PC (‘Crosslinked SLB’), or DOPC with 30 wt % 18:1 MPB PE (‘Crosslinked SLM’). The concentration of levofloxacin was maintained at 1 μg/mL. (B) The percentage of 1×106 THP-1 cells that remain viable upon continuous incubation with increasing concentrations of free levofloxacin, empty Fcγ-targeted protocells, or Fcγ-targeted, levofloxacin-loaded protocells for 48 hours. Cell viability was determined using propidium iodide and was normalized against untreated cells. Protocell SLBs were modified with 5 wt % of Fcγ and H5WYG (conjugated to DOPE, which was included in the SLB at 10 wt %) in all experiments. All experiments were conducted at 37° C. Data represent the mean±std. dev. for n=3. Unpublished data.

Supplementary FIG. 5. The biodistribution of protocells can be altered by controlling the size of the MSNP core. (A)-(C) 200 mg/kg of DyLight 633-labeled protocells or 100 μL of saline were injected into the tail veins of Balb/c mice, which were imaged with an IVIS Lumina II 1 hour (A), 48 hours (B), and 10 days (C) after injection. MSNP cores were 50, 150, or 250-nm in diameter, and SLBs were composed of DOPC with 30 wt % cholesterol and 10 wt % of PEG. Unpublished data.

Supplementary FIG. 6. Protocells do not aggregate in blood and can be passively excreted in the urine. (A)-(C) Fluorescence images of blood drawn from Balb/c mice 48 hours after being injected i.v. with 200 mg/kg of 250-nm (A), 150-nm (B), or 50-nm (C) DyLight 488-labeled protocells (green). Protocells remain well-dispersed, and their size differences are still evident relative to red blood cells (black). (D) TEM image of MSNP remnants present in the urine of a Balb/c mouse 24 hours after being injected i.v. with 200 mg/kg of 50-nm protocells. Largely intact MSNPs are visible, along with silica remnants. Unpublished data.

Supplementary FIG. 7. Schematic depicting intestinal penetration of orally-administered protocells. Protocells will be loaded in capsules coated with a polymer that will protect them from stomach pH but will dissolve in the ileum of the small intestine (pH 6.8-8.0). Protocells designed to be long-circulating (i) should rapidly enter circulation via the portal vein after being transcytosed by M cells. Fcγ-targeted protocells (ii and iii) are expected to be internalized by innate immune cells (e.g. macrophages, see Supplementary FIG. 11 for further details) in the lymphatic system; a portion should also enter circulation, however. Numbers in white circles refer to the steps in this process that address the first and third NATV objectives.

Supplementary FIG. 8. Schematic depicting the expected biodistributions of the three protocell formulations, as well as their expected states 12 hours after oral administration. Protocells designed to be long-circulating (i) should be systemically circulating by 12 hours, while Fcγ-targeted protocells (ii and iii) should be broadly distributed in various organs (lungs, liver, spleen, and lymph nodes) that are potential sites of Ft infection; as depicted in

Supplementary FIG. 7. A large portion of Fcγ-targeted protocells are expected to be internalized by innate immune cells in the lymphatic system, but some are expected to enter circulation via the portal vein as well. Fcγ-targeted protocells with sustained release profiles (ii) might start to release drug within 12 hours of administration, while Fcγ-targeted protocells with substrate-capped MSNPs (iii) should be in cytoplasm of target cells, as further depicted in Supplementary FIG. 11. Numbers in white circles refer to the steps in this process that address the second and third NATV objectives.

Supplementary FIG. 9. Schematic depicting the expected biodistributions of the three protocell formulations, as well as their expected states 72 hours after oral administration (i.e. at the time of SCHU S4 exposure). Protocells designed to be long-circulating (i) should largely still be systemically distributed after 72 hours, but some might start to accumulate within the liver and kidneys. The concentration of Fcγ-targeted protocells (ii and iii) within target tissues, including the lungs, liver, spleen, and lymph nodes, should reach a maximum by 72 hours. Protocells with sustained release profiles (i and ii) should start to release drug. Protocells intended to release drug in response to Ft infection (iii) should rapidly degrade in the presence of Ft (‘triggered’-see Supplementary FIG. 11 for additional details) but should stably retain drug in the absence of Ft (‘untriggered’). Numbers in white circles refer to the steps in this process that address the fourth and fifth NATV objectives.

Supplementary FIG. 10. Schematic depicting the expected biodistributions of the three protocell formulations, as well as their expected states 10 days after oral administration (i.e. one week after SCHU S4 exposure). All protocell formulations should still be detectable in the circulation and in various organs 10 days post-administration, albeit at a lower concentration than was present at 72 hours. Protocells designed for sustained release (i and ii) should slowly release encapsulated drug over a period of 10-14 days post-administration, while any untriggered particles (iii) should start to slowly degrade. Silica remnants should be benignly excreted through the urine and, to a lesser extent, the feces. We have shown protocells can persist in target tissues and in the liver for more than 4 weeks without causing any gross or histopathological toxicity.

Supplementary FIG. 11. Schematic depicting the interaction between Fcγ-targeted protocells and an Ft-infected innate immune cell that enables infection-triggered release of encapsulated antibiotic(s). Fcγ-targeted protocells are expected to efficiently bind to (1) and become internalized by (2) innate immune cells in a Fcγ receptor-mediated process. Phagosome acidification (3) is then expected to: (a) destabilize the SLB, exposing substrate-capped pores and (b) protonate phagosomolytic peptides present on the protocell SLB, which should disrupt phagosomal membranes and release antibiotic-loaded MSNPs into the cytosol (4). If Ft is present, specific host enzyme(s) expressed in response to infection (see Supplementary Table 3 for candidate enzymes and corresponding substrates) should degrade the substrate(s) capping the MSNP pores, which should trigger rapid dissolution of MSNP cores (5). The high localized concentration of antibiotic(s) should then readily kill intracellular Ft (6). Numbers in white circles refer to steps in this process that address the third, fourth, and fifth NATV objectives.

Supplementary FIG. 12. Schematic depicting the process we will use to construct protocells with substrate ‘stoppered’ MSNP pores and a tethered SLB. We will first soak MSNPs in a 10 mol % solution of the amine-containing silane, (3-aminopropyl)triethoxysilane (APTES) for 6 hours at room temperature. Aminated MSNPs (depicted in the upper left corner of the schematic) will then be reacted with a ‘membrane tether’ and an α-tosylated-ω-BOC-protected amino derivative of tri(ethyleneglycol), which will be deprotected via trifluoroacetic acid treatment to yield an amine-terminated tri(ethyleneglycol) ‘thread’ on the silica surface. We will then incubate MSNPs with a saturating concentration of levofloxacin for 4 hours at 4° C. and incubate levofloxacin-loaded protocells with α-cyclodextran (α-CD) for 24 hours at 4° C. After removing excess drug and α-CD via centrifugation, we will conjugate the primary amine moiety of the tri(ethyleneglycol) thread to a C-terminal cysteine residue, inserted in the substrate ‘stopper’ during its synthesis, using a commercially-available amine-to-sulfhydryl crosslinker. Finally, in order to accommodate the substrate stopper between the MSNP surface and SLB, we will use the ‘membrane tether’ to drive formation of a coherent SLB that is spaced ˜10 nm from the MSNP surface and anchored in place via cholesterol moieties in the tether molecule.

Supplementary FIG. 13. Cross-section of a protocell with substrate ‘stoppered’ pores and a tethered SLB. Protocells with drug-loaded, substrate-stoppered MSNP pores and a tethered SLB will be formed as depicted in Supplementary FIG. 12. The SLB will be composed of DOPC (or DSPC) with 30 wt % of cholesterol and 30 wt % of DOPE (or DSPE). 10 wt % of thiolated PEG-2000, 5 wt % of Fcγ (synthesized with a C-terminal cysteine residue), and 5 wt % of H5WYG (synthesized with a C-terminal cysteine residue) will then be conjugated to PE moieties using a commercially-available amine-to-sulfhydryl crosslinker with a (PEG)24 spacer. 10 wt % of the RGD peptide will be conjugated to PE moieties by its C-terminus using an acid-labile crosslinker that dissociates at pH 5.0; 35 this crosslinker contains a PEG spacer as well, the length (n) of which can be modulated to ensure that the RGD peptide is surface-exposed. R in both of the crosslinker structures denotes the PE headgroup, while R′ in the lower crosslinker structure denotes PEG, Fcγ, or H5WYG.

Supplementary FIG. 14. Intravital imaging of ex ovo avian embryos enables the spatiotemporal dynamics of nanoparticles to be monitored in real-time and with single-cell resolution. (A) Image showing an ex ovo yolk sac (yellow), embryo (magenta), and highly vascularized CAM (red) 7 days after fertilization. The embryo's eye is indicated by the white arrow. (B)-(C) Grey-scale fluorescence images of blood vessels (whitish-grey) in the CAM, which were injected with FITC for contrast. Individual endothelial and red blood cells (small black areas) can be seen in (C). The inset of (C) shows a bright-field image of red blood cells (grey discs) flowing in a venule. (D) Falsely-colored fluorescence image of the CAM capillary bed, showing 50-nm protocells (green) and 250-nm protocells (red) flowing in an arteriole (A) and a venule (V). White arrows indicate the direction of blood flow from the arteriole, through the capillary bed, to the venule. Tissue autofluorescence is shown in blue, and red blood cells are visible as dark areas (one such area is indicated by the yellow asterisk). It is important to note that (B)-(C) show a three-dimensional tissue. Unpublished data.

Supplementary FIG. 15. In vivo targeting efficacy can be assessed in ex ovo avian embryos. (A) Grey-scale fluorescence image of Fcγ-targeted, DyLight 488-labeled protocells (white spots) accumulating at the surface of an innate immune cell (indicated by the white circle) after injection of protocells into the CAM. The ‘A’ indicates an arteriole, and the ‘V’ indicates a venule. White arrows indicate the direction of blood flow in the vessels (black). (B) SEM image of a venule from a fixed, freeze-fractured avian embryo. An innate immune cell (indicated by the white circle) is visible amongst the disc-shaped red blood cells.

### DETAILED DESCRIPTION OF THE INVENTION

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal (eg. dog, cat, horse, cow, pig, sheep, goat, bird, etc.) and preferably a human, to whom treatment, including prophylactic treatment (prophylaxis), with the compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis (which results in a reduced likelihood of an infection) and/or therapy of an infection and/or disease state or as otherwise described herein. The term effective subsumes all other effective amount or effective concentration terms (including the term “therapeutically effective”) which are otherwise described or used in the present application.

The term “Mycobacterium”, is used to describe a genus of Actinobacteria, given its own family, the Mycobacteriaceae. The genus includes pathogens known to cause serious diseases in mammals, including tuberculosis and leprosy. The Latin prefix “myco” means both fungus and wax; its use here relates to the “waxy” compounds in the cell wall. Mycobacteria are aerobic and non-motile bacteria (except for the species Mycobacterium marinum which has been shown to be motile within macrophages) that are characteristically acid-alcohol fast. Mycobacteria do not contain endospores or capsules, and are usually considered Gram-positive. While mycobacteria do not seem to fit the Gram-positive category from an empirical standpoint (i.e. they do not retain the crystal violet stain), they are classified as an acid-fast Gram-positive bacterium due to their lack of an outer cell membrane. All Mycobacterium species share a characteristic cell wall, thicker than in many other bacteria, which is hydrophobic, waxy, and rich in mycolic acids/mycolates. The cell wall makes a substantial contribution to the hardiness of this genus.

Many Mycobacterium species adapt readily to growth on very simple substrates, using ammonia or amino acids as nitrogen sources and glycerol as a carbon source in the presence of mineral salts. Optimum growth temperatures vary widely according to the species and range from 25° C. to over 50° C.

Some species can be very difficult to culture (i.e. they are fastidious), sometimes taking over two years to develop in culture. Further, some species also have extremely long reproductive cycles: M. leprae (leprosy), may take more than 20 days to proceed through one division cycle (for comparison, some E. coli strains take only 20 minutes), making laboratory culture a slow process.

A natural division occurs between slowly—and rapidly—growing species. Mycobacteria that form colonies clearly visible to the naked eye within 7 days on subculture are termed rapid growers, while those requiring longer periods are termed slow growers. Mycobacteria are slightly curved or straight rods between 0.2-0.6 μm wide by 1.0-10 μm long.

A “Mycobacterium infection” includes, but is not limited to, tuberculosis and atypical mycobacterial infections cause by a Mycobacterium species other than M. tuberculosis. Atypical mycobacterial infections include, but are not limited to, abscesses, septic arthritis, and osteomyelitis (bone infection). They can also infect the lungs, lymph nodes, gastrointestinal tract, skin, and soft tissues. Atypical mycobacterial infections can be caused by Mycobacterium avium-intracellulare, which frequently affects AIDS patients and causes lung disease. Mycobacterium marinum cause skin infections and is also responsible for swimming pool granuloma. Mycobacterium ulcerans cause skin infections. Mycobacterium kansasii causes lung disease.

A particularly important Mycobacterium species to the present invention is M. tuberculosis. The term “Tuberculosis” or “TB” is used to describe the infection caused by the infective agent “Mycobacterium tuberculosis” or “M. tuberculosis”, a tubercle bacillus bacteria. Tuberculosis is a potentially fatal contagious disease that can affect almost any part of the body but is most frequently an infection of the lungs. It is caused by a bacterial microorganism, the tubercle bacillus or Mycobacterium tuberculosis.

Tuberculosis is primarily an infection of the lungs, but any organ system is susceptible, so its manifestations may be varied. Effective therapy and methods of control and prevention of tuberculosis have been developed, but the disease remains a major cause of mortality and morbidity throughout the world. The treatment of tuberculosis has been complicated by the emergence of drug-resistant organisms, including multiple-drug-resistant tuberculosis, especially in those with HIV infection.

Mycobacterium tuberculosis, the causative agent of tuberculosis, is transmitted by airborne droplet nuclei produced when an individual with active disease coughs, speaks, or sneezes. When inhaled, the droplet nuclei reach the alveoli of the lung. In susceptible individuals the organisms may then multiply and spread through lymphatics to the lymph nodes, and through the bloodstream to other sites such as the lung apices, bone marrow, kidneys, and meninges.

The development of acquired immunity in 2 to 10 weeks results in a halt to bacterial multiplication. Lesions heal and the individual remains asymptomatic. Such an individual is said to have tuberculous infection without disease, and will show a positive tuberculin test. The risk of developing active disease with clinical symptoms and positive cultures for the tubercle bacillus diminishes with time and may never occur, but is a lifelong risk. Approximately 5% of individuals with tuberculous infection progress to active disease. Progression occurs mainly in the first 2 years after infection; household contacts and the newly infected are thus at risk.

Many of the symptoms of tuberculosis, whether pulmonary disease or extrapulmonary disease, are nonspecific. Fatigue or tiredness, weight loss, fever, and loss of appetite may be present for months. A fever of unknown origin may be the sole indication of tuberculosis, or an individual may have an acute influenza-like illness. Erythema nodosum, a skin lesion, is occasionally associated with the disease.

The lung is the most common location for a focus of infection to flare into active disease with the acceleration of the growth of organisms. Infections in the lung are the primary focus of the present invention. There may be complaints of cough, which can produce sputum containing mucus, pus- and, rarely, blood. Listening to the lungs may disclose rales or crackles and signs of pleural effusion (the escape of fluid into the lungs) or consolidation if present. In many, especially those with small infiltration, the physical examination of the chest reveals no abnormalities.

Miliary tuberculosis is a variant that results from the blood-borne dissemination of a great number of organisms resulting in the simultaneous seeding of many organ systems. The meninges, liver, bone marrow, spleen, and genitourinary system are usually involved. The term miliary refers to the lung lesions being the size of millet seeds (about 0.08 in. or 2 mm). These lung lesions are present bilaterally. Symptoms are variable.

Extrapulmonary tuberculosis is much less common than pulmonary disease. However, in individuals with AIDS, extrapulmonary tuberculosis predominates, particularly with lymph node involvement, with some pulmonary impact. For example, fluid in the lungs and lung lesions are other common manifestations of tuberculosis in AIDS. The lung is the portal of entry, and an extrapulmonary focus, seeded at the time of infection, breaks down with disease occurring.

Development of renal tuberculosis can result in symptoms of burning on urination, and blood and white cells in the urine; or the individual may be asymptomatic. The symptoms of tuberculous meningitis are nonspecific, with acute or chronic fever, headache, irritability, and malaise.

A tuberculous pleural effusion can occur without obvious lung involvement. Fever and chest pain upon breathing are common symptoms. Bone and joint involvement results in pain and fever at the joint site. The most common complaint is a chronic arthritis usually localized to one joint. Osteomyelitis is also usually present. Pericardial inflammation with fluid accumulation or constriction of the heart chambers secondary to pericardial scarring are two other forms of extrapulmonary disease.

At present, the principal methods of diagnosis for pulmonary tuberculosis are the tuberculin skin test (an intracutaneous injection of purified protein derivative tuberculin is performed, and the injection site examined for reactivity), sputum smear and culture, and the chest x-ray. Culture and biopsy are important in making the diagnosis in extrapulmonary disease.

A combination of two or more drugs is often used in the initial traditional therapy of tuberculous disease. Drug combinations are used to lessen the chance of drug-resistant organisms surviving. The preferred treatment regimen for both pulmonary and extrapulmonary tuberculosis is a 6-month regimen of the antibiotics isoniazid, rifampin, and pyrazinamide given for 2 months, followed by isoniazid and rifampin for 4 months. Because of the problem of drug-resistant cases, ethambutol can be included in the initial regimen until the results of drug susceptibility studies are known. Once treatment is started, improvement occurs in almost all individuals. Any treatment failure or individual relapse is usually due to drug-resistant organisms.

In certain non-limiting embodiments, a protocell-treated host cell is infected by one or more bacteria selected from the group consisting of Yersinia pestis, Bacillus anthracis, Francisella tularensis, Burkholderia pseudomallei, Burkholderia mallei, Escherichia coli, Pseudomonas aeruginosa, Plasmodium falciparum, Mycobacterium tuberculosis, vancomycin-resistant enterococci (VRE), methicillin-resistant Staphylococcus aureus (MRSA), glycopeptide intermediate susceptible Staphylococcus aureus (GISA), coagulase-negative staphylococci (CNS), and penicillin-resistant Streptococcus pneumoniae (PRSP), enteropathogenic E. coli (EPEC), enterotoxigenic E. coli (ETEC), enterinvasive E. coli (EIEC), enterohemorrhagic E. coli (EHEC) and Clostridium difficile.

In certain non-limiting embodiments, a protocell-treated host cell is infected by one or more bacteria selected from the group consisting of Enterobacteriaceae, in particular Escherichia, Salmonella, Shigella, Citrobacter, Edwardsiella, Enterobacter, Hafnia, Klebsiella, Morganella, Proteus, Providencia, Serratia, and Yersinia, Pseudomonadaceae, in particular Pseudomonas, Burkholderia, Stenotrophomonas, Shewanella, Sphingomonas and Comamonas, Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae, in particular Treponema and Borrelia, Leptospiraceae, Campylobacter, Helicobacter, Spirillum, Streptobacillus, Bacteroidaceae, in particular Bacteroides, Fusobacterium, Prevotella and Porphyromonas, and Acinetobacter, in particular A. baumanii; and/or Gram-positive bacteria selected from the group consisting of Listeria monocytogenes, Staphylococcus aureus, Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium tuberculosis, Corynebacterium diphteriae, Mycoplasma pneumoniae, and Actinomyces.

In certain non-limiting embodiments, a protocell-treated host cell is infected by one or more biological warfare agents selected from the group consisting of Bacillus anthracis (anthrax), Burkholderia mallei (glanders), Burkholderia pseudomallei (melioidosis), Clostridium botulinum toxin (botulism), Francisella tularensis (tularemia), Vibrio cholerae (cholera) and Yersinia pestis (plague).

In certain non-limiting embodiments, protocells of the invention comprise or are co-administered with one or more antibiotics selected from the group consisting of rifampicin, oxacillin, ampicillin, b-lactam antibiotics, rifamycin group antibiotics, ciprofloxacin, erythromycin, macrolides, methicillin, metronidazole, ofloxacin, penicillin, streptomycin, tetracycline and vancomycin.

In certain non-limiting embodiments, protocells of the invention comprise or are co-administered with one or more antibiotics selected from the group consisting of streptomycin, chloramphenicol, tetracyclines, sulfonamides (e.g., trimethoprim-sulfamethoxazole), gentamicin, doxycycline, and fluoroquinolones (e.g., ciprofloxacin).

In certain non-limiting embodiments, protocells of the invention comprise or are co-administered with one or more antibiotics selected from the group consisting of fluoroquinolones (preferably ciprofloxacin (cipro)), tetracyclines (preferably doxycycline), erythromycin, vancomycin and penicillin.

The term “compound” is used herein to describe any specific compound or bioactive agent disclosed herein, including any and all stereoisomers (including diasteromers), individual optical isomers (enantiomers) or racemic mixtures, isotopologues, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment of the present invention. Exemplary bioactive agents include the compounds according to the present invention which are used to treat F. tularensis, B. pseudomallei and P. aeruginosa-related infections.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, prevention/preventive treatment, delay in or inhibition of the likelihood of the onset of the disease, etc. In the case of F. tularensis, B. pseudomallei or P. aeruginosa-related infections or infections associated with the other bacteria described herein, these terms also apply to bacterial infections and preferably include, in certain particularly favorable embodiments the eradication or elimination (as provided by limits of diagnostics) of the bacterium which is the causative agent of the infection. Similarly, infections caused the other bacteria mentioned above are also treated by protocells, formulations and methods of the invention.

Treatment, as used herein, encompasses both prophylactic and therapeutic treatment. Compositions according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs. Alternatively, compounds according to the present invention can, for example, be administered therapeutically to a mammal that is already afflicted by disease. Administration of the compositions according to the present invention is effective to decrease the severity of the disease or lengthen the lifespan of the mammal so afflicted, e.g. as in the case of a F. tularensis, B. pseudomallei or P. aeruginosa-related infection or infections associated with the other bacteria described herein, or inhibit or even eliminate the causative agent of the disease.

“Antibiotics” include, but are not limited to, compositions selected from the group consisting of Gentamicin, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Cephalothin, Cephalexin, Cefaclor, Cefamandole, Cefoxitin, Cefprozil, Cefuroxime, Cefixime, Cefdinir, Cefditoren, Cefoperazone Cefotaxime, Cefpodoxime, Ceftazadime, Ceftibuten, Ceftizoxime Ceftriaxone, Cefepime, Ceftaroline fosamil, Ceftobiprole, Teicoplanin, Vancomycin, Telavancin, Daptomycin, Oritavancin, WAP-8294A, Azithromycin, Clarithromycin, Dirithromycin, Erythromycin, Roxithromycin, Telithromycin, Spiramycin, Clindamycin, Lincomycin, Aztreonam, Furazolidone, Nitrofurantoin, Oxazolidonones, Linezolid, Posizolid, Radezolid, Torezolid, Amoxicillin, Ampicillin, Azlocillin, Carbenicillin, Cloxacillin Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Demeclocycline, Doxycycline, Vibramycin Minocycline, Tigecycline, Oxytetracycline, Tetracycline, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing (preventive treatment of) a disease, condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients or subjects, administration with compounds according to the present invention will reduce the likelihood or inhibit a particular condition or disease state (e.g. the worsening of a F. tularensis, B. pseudomallei or P. aeruginosa-related infection) or other accepted indicators of disease progression from occurring.

The term “protocell” is used to describe a porous nanoparticle which is made of a material comprising silica and optional additional components including polystyrene, alumina, titania, zirconia, or generally metal oxides, organometallates, organosilicates or mixtures thereof.

In certain embodiments, the porous particle core may be hydrophilic and can be further treated to provide a more hydrophilic surface in order to influence pharmacological result in a particular treatment modality. For example, mesoporous silica particles according to the present invention can be further treated with, for example, ammonium hydroxide or other bases and hydrogen peroxide to provide significant hydrophilicity. The use of amine containing silanes such as 3-[2-(2-aminoethylamino)ethylamino]propyltrimethoxysilane (AEPTMS), among others, may be used to produce negatively charged cores which can markedly influence the cargo loading of the particles. Other agents may be used to produce positively charged cores to influence in the cargo in other instances, depending upon the physicochemical characteristics of the cargo.

In certain embodiments, the PEGylated lipid bilayer includes a phospholipid (which may be PEGylated) and may also comprise cholesterol (for structural integrity of the lipid bilayer which itself may be PEGylated) as well as polyethylene glycol lubricants/solvents (e.g. PEG 2000, etc.) to provide flexibility to the lipid bilayer. In addition to fusing a single phospholipid bilayer, multiple bilayers with opposite charges may be fused onto the porous particles in order to further influence cargo loading, sealing and release of the particle contents in a biological system.

In certain embodiments, the lipid bilayer can be prepared, for example, by extrusion of hydrated lipid films through a filter of varying pore size (e.g., 50, 100, 200 nm) to provide filtered lipid bilayer films, which can be fused with the porous particle cores, for example, by pipette mixing or other standard method.

In various embodiments, the protocell can be loaded with and seal macromolecules (siRNAs and polypeptide toxins) as otherwise described herein, thus creating a loaded protocell useful for cargo delivery across the cell membrane

In preferred aspects of the present invention, the protocells provide a targeted delivery through conjugation of certain targeting peptides onto the protocell surface, preferably by conjugation to the lipid bilayer surface. These peptides may be synthesized for example, with C-terminal cysteinyl residues and conjugated to one or more of the phospholipids (especially DOPE, which contains a phosphoethanolamine group) which comprise the lipid bilayer. Other approaches for linking a targeting peptide to a functional group in one or more phospholipids (depending on the lipids used to form the bilayer/multilayer of protocells according to the present invention) which are used in the present invention are readily recognized by those of ordinary skill in the art.

The porous particle, such as porous silica particles, can be surface charged. For example, the surface charge of the porous silica particles can switch from negative to positive at neutral pHs by using amine-modified silane precursors and controlling the percentage of amine groups within the porous silica particles. For example, the porous silica particles can have a composition of about 5% to about 50% amine, such as about 10% to about 50% amine, or about 5% to about 30% amine by weight; and the amine-modified silane precursors can include, for example,

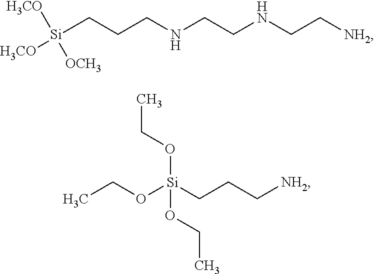 [SMILES: CO[Si](CCCNCCNCCN)(OC)OC ; CCO[Si](CCCN)(OCC)OCC]

etc.

The porous silica particles can be formed by, for example, mixing water, HCl, ethanol, cetyltrimethylammonium bromide (CTAB), and tetraethyl orthosilicate (TEOS), as disclosed in a related International Patent Application No. PCT/US10/20096, entitled “Porous Nanoparticle Supported Lipid Bilayer Nanostructures,” which is hereby incorporated by reference in its entirety.

As described above, charge and/or hydrophobicity of the mesoporous silica nanoparticle can be varied by addition of one or more aminosilanes and/or silyl group capping agents depending upon the charge and/or hydrophobicity of the one or more antibiotics. Aminosilanes can be selected from the group consisting of (3-aminopropyl)triethoxysilane (APTES), p-aminophenyltrimethoxysilane, p-aminophenyltrimethoxysilane, N-phenylaminopropyltrimethoxysilane N-phenylaminopropyltriethoxysilane, n-butylaminopropyltrimethoxysilane, n-butylaminopropyltriethoxysilane, 3-(N-allylamino)propyltrimethoxysilane, (N,N-diethyl-3-aminopropyl)trimethoxysilane, and (N,N-diethyl-3-aminopropyl) triethoxysilane. Silyl group capping agent can be selected from the group consisting of 1,1,1,3,3,3-hexamethyldisilazane (HMDS), trimethylmethoxysilane, phenyldimethylmethoxysilane and octyldimethylmethoxysilane.

The porous nanoparticulates can also comprise one or more compositions selected from the group consisting of aliphatic polyesters, poly (lactic acid) (PLA), poly(glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), poly(acrylic acid), alginate and other polysaccharides, collagen, and chemical derivatives thereof, albumin a hydrophilic protein, zein, a prolamine, a hydrophobic protein, and copolymers and mixtures thereof, among others well known in the art.

A porous spherical silica nanoparticle is used for the preferred protocells and is surrounded by a supported lipid or polymer bi-layer or multilayer. Various embodiments according to the present invention provide nanostructures and methods for constructing and using the nanostructures and providing protocells according to the present invention. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from Melorium Technologies, Rochester, N.Y. SkySpring Nanomaterials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009). Protocells can be readily obtained using methodologies known in the art. The examples section of the present application provides certain methodology for obtaining protocells which are useful in the present invention. Protocells according to the present invention may be readily prepared, including protocells comprising lipids which are fused to the surface of the silica nanoparticle. See, for example, Liu, et al., Chem. Comm., 5100-5102 (2009), Liu, et al., J. Amer. Chem. Soc., 131, 1354-1355 (2009), Liu, et al., J. Amer. Chem. Soc., 131, 7567-7569 (2009) Lu, et al., Nature, 398, 223-226 (1999), Preferred protocells for use in the present invention are prepared according to the procedures which are presented in Ashley, et al., Nature Materials, 2011, May; 10(5):389-97, Lu, et al., Nature, 398, 223-226 (1999), Caroll, et al., Langmuir, 25, 13540-13544 (2009), and as otherwise presented in the experimental section which follows.

Covalent attachment of lipid bilayer components (especially including cholesterol and/or phospholipids) to the nanoparticle surface is accomplished via the reaction of heterobifunctional crosslinkers towards functionalized silanes (described herein) on the silica surface followed by reaction with either functionalized phospholipid or cholesterol molecules and electrostatic SLB fusion. In both cases, the silica is functionalized with an organosilane containing a reactive group (i.e. glycidoxypropylsilane or APTES). Particles functionalized with glycidoxypropyl silane are mixed with liposomes containing cholesterol modified with a PEG spacer and primary hydroxyl group which will react with the glycidoxy functionalized silica. Particles functionalized with APTES are mixed with liposomes containing DPPC thioethanol that have been conjugated to a heterobifunctional crosslinker reactive towards sulfhydryls and primary amines. In all cases the liposomes are also formed with DOPC, DOPG or DOPS, and cholesterol.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, or a whisker. A nanoparticle may include particles having two or more of the aforementioned shapes; In one embodiment, a cross-sectional geometry of the particle may be one or more of circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The phrase “effective average particle size” as used herein to describe a multiparticulate (e.g., a porous nanoparticulate) means that at least 50% of the particles, therein are of a specified size. Accordingly, “effective average particle size of less than about 2,000 nm in diameter” means that at least 50% of the particles therein are less than about 2000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. “D₅₀” refers to the particle size below which 50% of the particles in a multiparticulate fall. Similarly, “D₉₀” is the particle size below which 90% of the particles in a multiparticulate fall.

In certain embodiments, the porous nanoparticulates are comprised of one or more compositions selected from the group consisting of silica, a biodegradable polymer, a solgel, a metal and a metal oxide.

Mesopores (IUPAC definition 2 nm to 50 nm in diameter and distinguished from the present invention which ranges from about 1 nm to about 75 nm unless indicated) are molded or formed by templating agents including surfactants, block copolymers, molecules, macromolecules, emulsions, latex beads or nanoparticles. In one embodiment, core particles of generally spherical shape are formed by generating an aerosol dispersion of the templating agent and the core material (e.g., aluminum chloride (AlCl₃.6H₂O) for aluminum hydroxide nanoparticle; alum (KAl(SO₄)₂.12H₂O) for aluminum sulfate nanoparticle; and tetraethyl orthosilicate (TEOS) for a silica nanoparticle) in a tubular reactor and then drying the particles. See, e.g., “Aerosol-assisted self-assembly of mesostructured spherical nanoparticles,” Lu, Y., et al., Nature, Vol. 398, 223-26 (1999), incorporated herein by reference. Generally, in an aerosol-assisted evaporation-induced self-assembly (EISA) process, a dilute solution of a metal salt or metal alkoxide is dissolved in an alcohol/water solvent along with ionic or non-ionic surfactants, block copolymers (e.g., Pluronic P-123, a triblock copolymer manufactured by BASF Corporation). The resulting solution is then aerosolized with a carrier gas and introduced into a laminar flow reactor. Surfactants/templates can be extracted using either acidified ethanol or thermal calcination to yield mesoporous hydroxides (boehmite AlO(OH) or gibbsite Al(OH)₃) or sulfates (alum). The process may be used to form particles with systemically variable pore sizes (e.g., 2 nm to 50 nm), pore geometrics (e.g., hexagonal, cubic, lamellar, cellular) and surface areas (100 m²/g to greater than 1200 m²/g).

In certain embodiments, the cargo components can include, but are not limited to, chemical small molecules (especially antibiotics, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In other embodiments, the lipid bilayer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

The protocells particle size distribution, according to the present invention, depending on the application, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (i.e., a polydisperse population can vary widely from a mean or medium diameter, e.g., up to ±200-nm or more if prepared by aerosol. Polydisperse populations can be sized into monodisperse populations. All of these are suitable for protocell formation. In the present invention, preferred protocells are preferably no more than about 500 nm in diameter, preferably no more than about 200 nm in diameter in order to afford delivery to a patient or subject and produce an intended therapeutic effect.

In one embodiment, the present invention is directed to high surface area (i.e., greater than about 600 m²/g, preferably about 600 to about 1,000-1,250 mg²/g), preferably monodisperse spherical silica or other biocompatible material nanoparticles having diameters falling within the range of about 0.05 to 50 μm, preferably about 1,000 nm or less, more preferably about 100 nm or less, 10-20 nm in diameter, a multimodal pore morphology comprising large (about 1-100 nm, preferably about 2-50 nm, more preferably about 10-35 nm, about 20-30 nm) surface-accessible pores interconnected by smaller internal pores (about 2-20 nm, preferably about 5-15 nm, more preferably about 6-12 nm) volume, each nanoparticle comprising a lipid bilayer (preferably a phospholipid bilayer) supported by said nanoparticles (the phospholipic bilayer and silica nanoparticles together are labeled “protocells”), to which is bound at least one antigen which binds to a targeting polypeptide or protein on a cell to which the protocells are to be targeted, wherein the protocells further comprise (are loaded) with a small molecule anticancer agent and/or a small interfering RNA (siRNA).

The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi, et al., Advanced Materials, 2005, 17, No. 8, 1067-1072.

In certain embodiments, protocells according to the present invention generally range in size from greater than about 8-10 nm to about 5 μm in diameter, preferably about 20-nm-3 μm in diameter, about 10 nm to about 500 nm or more, more preferably about 20-200-nm (including about 150 nm, which may be a mean or median diameter), depending on the size of the nanoparticle as well as the number of lipid layers (bilayer or multilayer) which coat the nanoparticle. As discussed above, the protocell population may be considered monodisperse or polydisperse based upon the mean or median diameter of the population of protocells. Size is an important aspect to therapeutic and diagnostic efficacy of the present invention as particles smaller than about 8-nm diameter are excreted through kidneys, and those particles larger than about 200 nm are trapped by the liver and spleen. Thus, an embodiment of the present invention focuses on smaller sized protocells for drug delivery and diagnostics in the patient or subject.

Pore surface chemistry of the nanoparticle material can be very diverse—all organosilanes yielding cationic, anionic, hydrophilic, hydrophobic, reactive groups—pore surface chemistry, especially charge and hydrophobicity, affect loading capacity. Attractive electrostatic interactions or hydrophobic interactions control/enhance loading capacity and control release rates. Higher surface areas can lead to higher loadings of drugs/cargoes through these attractive interactions.

In certain embodiments, the surface area of nanoparticles, as measured by the N2 BET method, ranges from about 100 m²/g to >about 1200 m²/g. In general, the larger the pore size, the smaller the total surface area. The surface area theoretically could be reduced to essentially zero, if one does not remove the templating agent or if the pores are sub-0.5-nm and therefore not measurable by N2 sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

The charge of the mesoporous silica nanoparticulate core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Typically the protocells according to the present invention are loaded with cargo to a capacity up to about 50 weight %: defined as (cargo weight/weight of loaded protocell)×100. The loading of cargo is often about 0.01 to 10% but this depends on the drug or drug combination which is incorporated as cargo into the protocell. This is generally expressed in μM per 10¹⁰ particles where we have values ranging from 2000-100 μM per 10¹⁰ particles. Preferred protocells according to the present invention exhibit release of cargo at pH about 5.5, which is that of the endosome, but are stable at physicological pH of 7 or higher (7.4).

The surface area of the internal space for loading is the pore volume whose optimal value ranges from about 1.1 to 0.5 cubic centimeters per gram (cc/g). Note that in the protocells according to one embodiment of the present invention, the surface area is mainly internal as opposed to the external geometric surface area of the nanoparticle.

The lipid bilayer supported on the porous particle according to one embodiment of the present invention has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

Numerous lipids which are used in liposome delivery systems may be used to form the lipid bilayer on nanoparticles to provide protocells according to the present invention. Virtually any lipid which is used to form a liposome may be used in the lipid bilayer which surrounds the nanoparticles to form protocells according to an embodiment of the present invention. Preferred lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Other lipids for use in the present invention include 1,2-distearoyl-sn-glycero-3-phosphoethanolamine (DSPE), 1-palmitoyl-2-oleoyl-sn-glycero-3-phosphoethanolamine (POPE), phosphatidylethanolamine (PE), monomethyl-phosphatidylethanolamine (MMPE) and dimethyl-phosphatidylethanolamine (DMPE), each of which may be pegylated for use in the present invention. Cholesterol also may be included and in certain embodiments is an important component of the lipid bilayer of protocells according to an embodiment of the invention. Often cholesterol is incorporated into lipid bilayers of protocells in order to enhance structural integrity of the bilayer. These lipids are all readily available commercially from Avanti Polar Lipids, Inc. (Alabaster, Ala., USA). Of the above lipids, DOPE, DPPE, DSPE, MMPE, DMPE, PE and POPE, are particularly useful for conjugating (through an appropriate crosslinker) peptides, polypeptides, including immunogenic peptides, proteins and antibodies, RNA and DNA through the amine group on the lipid.

As discussed, the lipid bilayer can be PEGylated with a variety of polyethylene glycol-containing compositions. PEG molecules can have a variety of lengths and molecular weights and include, but are not limited to, PEG 200, PEG 1000, PEG 1500, PEG 4600, PEG 10,000, PEG-peptide conjugates or combinations thereof. In preferred aspects, pegylated lipids including PEGylated DOPE, PEGylated DPPE, PEGylated DSPE, PEGylated DMPE, PEGylated MMPE, PEGylated PE, PEGylated POPE, among others may be used as the PEGylated lipid component in lipid bilayers used in the present invention. In alternative embodiments, cholesterol may be pegylated by linking a PEG group with the hydroxyl of the cholesterol moiety through a linking group.

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

The term “reporter” is used to describe an imaging agent or moiety which is incorporated into the phospholipid bilayer or core cargo according to an embodiment of the present invention and provides a signal which can be measured. The moiety may provide a fluorescent signal or may be a radioisotope which allows radiation detection, among others. Exemplary fluorescent labels for use in protocells (preferably via conjugation or adsorption to the lipid bilayer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moities which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of an infection (infected tissue) in a patient and or the progress of therapy in a patient or subject.

The term “histone-packaged supercoiled plasmid DNA” is used to describe an optional component of protocells according to the present invention which utilize a preferred plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein, which may be helpful in treating a microbial infection and/or the secondary effects of such an infection. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g. NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are preferably used. In certain aspects of the invention, a combination of human histone proteins H1, H2A, H2B, H3 and H4 in a preferred ratio of 1:2:2:2:2, although other histone proteins may be used in other, similar ratios, as is known in the art or may be readily practiced pursuant to the teachings of the present invention. The DNA may also be double stranded linear DNA, instead of plasmid DNA, which also may be optionally supercoiled and/or packaged with histones or other packaging components.

Other histone proteins which may be used in this aspect of the invention include, for example, H1F, H1F0, H1FNT, H1FOO, H1FX H1H1 HIST1H1A, HIST1H1B, HIST1H1C, HIST1H1D, HIST1H1E, HIST1H1T; H2AF, H2AFB1, H2AFB2, H2AFB3, H2AFJ, H2AFV, H2AFX, H2AFY, H2AFY2, H2AFZ, 112A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells according to the present invention may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver immunogenic, therapeutic and/or diagnostic molecules (polypeptide and/or nucleotide) into the nucleus of the targeted cell. Any number of crosslinking agents, well known in the art, may be used to covalently link an antigenic peptide to the lipid bilayer or other components of the protocells, or a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Preferred nuclear localization sequences include H₂N-GNQSSNFGPMKGGNFGGRSSGPYGGGGQYFAKPRNQGGYGGC—COOH SEQ I.D NO: 1, RRMKWKK (SEQ ID NO:2), PKKKRKV (SEQ ID NO:3), and KR[PAATKKAGQA]KKKK (SEQ ID NO:4), the NLS of nucleoplasmin, a prototypical bipartite signal comprising two clusters of basic amino acids, separated by a spacer of about 10 amino acids. Numerous other nuclear localization sequences are well known in the art. See, for example, LaCasse, et al., Nuclear localization signals overlap DNA- or RNA-binding domains in nucleic acid-binding proteins. Nucl. Acids Res., 23, 1647-16561995); Weis, K. Importins and exportins: how to get in and out of the nucleus [published erratum appears in Trends Biochem Sci 1998 July; 23(7):235]. TIBS, 23, 185-9 (1998); and Murat Cokol, Raj Nair & Burkhard Rost, “Finding nuclear localization signals”, at the website ubic.bioc.columbia.edu/papers/2000 nls/paper.html#tab2.

The terms “co-administer” and “co-administration” are used synonymously to describe the administration of at least one of the compositions according to the present invention in combination with at least one other agent (in nanoparticles or outside of nanoparticles or even in separate pharmaceutical compositions), often at least one additional antibiotic or antiviral agent (as otherwise described herein), which are specifically disclosed herein in amounts or at concentrations which would be considered to be effective amounts at or about the same time. While it is preferred that co-administered compositions/agents be administered at the same time, agents may be administered at times such that effective concentrations of both (or more) compositions/agents appear in the patient at the same time for at least a brief period of time. Alternatively, in certain aspects of the present invention, it may be possible to have each co-administered composition/agent exhibit its inhibitory effect at different times in the patient, with the ultimate result being the inhibition and treatment of an infection, as well as the reduction or inhibition of other (including secondary) disease states, conditions or complications. Of course, when more than one disease state, infection or other condition is present, the present compounds may be combined with other agents to treat that other infection or disease or condition as required.

The terms “targeting ligand” and “targeting active species” are used to describe a compound or moiety (preferably an antigen) which is complexed or preferably covalently bonded to the surface of a protocell according to the present invention which binds to a moiety on the surface of a cell to be targeted so that the protocell may selectively bind to the surface of the targeted cell and deposit its contents into the cell. The targeting active species for use in the present invention is preferably a targeting peptide as otherwise described herein, a polypeptide including an antibody or antibody fragment, an aptamer, or a carbohydrate, among other species which bind to a targeted cell.

A “targeting peptide” is one type of targeting ligand and is a peptide which binds to a receptor or other polypeptide in a target cell (e.g. an infected cell) and allows the targeting of protocell according to the present invention to particular cells which express a peptide (be it a receptor or other functional polypeptide) to which the targeting peptide binds. Targeting peptides may be complexed or preferably, covalently linked to the lipid bilayer through use of a crosslinking agent as otherwise described herein.

The terms “fusogenic peptide” and “endosomolytic peptide” are used synonymously to describe a peptide which is optionally and preferred crosslinked onto the lipid bilayer surface of the protocells according to the present invention. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative and preferred fusogenic peptides for use in protocells according to the present invention include H5WYG peptide, H₂N—GLFHAIAHFIHGGWHGLIHGWYGGC—COOH (SEQ ID NO:5) or a mer polyarginine (H₂N—RRRRRRRR—COOH, SEQ ID NO:6) among others known in the art.

The term “cross-linking agent” or “linking agent” is used to describe a compound which may be used to covalently link various components according to the present invention to each other, such as a bifunctional compound of varying length containing two different functional groups. Crosslinking agents according to the present invention may contain two electrophilic groups (to react with nucleophilic groups on peptides of oligonucleotides, one electrophilic group and one nucleophilic group or two two nucleophilic groups). The crosslinking agents may vary in length depending upon the components to be linked and the relative flexibility required. Crosslinking agents are used to anchor targeting and/or fusogenic peptides to the phospholipid bilayer, to link nuclear localization sequences to histone proteins for packaging supercoiled plasmid DNA and in certain instances, to crosslink lipids in the lipid bilayer of the protocells. There are a large number of crosslinking agents which may be used in the present invention, many commercially available or available in the literature. Preferred crosslinking agents for use in the present invention include, for example, 1-Ethyl-3-[3-dimethylaminopropyl]carbodiimide hydrochloride (EDC), succinimidyl 4[N-maleimidomethyl]cyclohexane-1-carboxylate (SMCC), N-[β-Maleimidopropionic acid] hydrazide (BMPH), NHS-(PEG)ₙ-maleimide, succinimidyl-[(N-maleimidopropionamido)-tetracosaethyleneglycol]ester (SM(PEG)₂₄), and succinimidyl 6-[3′-(2-pyridyldithio)-propionamido] hexanoate (LC-SPDP), among others.

In one embodiment, the present invention may include an immunogen in order to induce both humoral and cellular immune responses and to tune the magnitude of an immunogenic response.

As explained above, another aspect of the invention relates to the use of aerosol-assisted evaporation-induced self-assembly to provide mesoporous nanoparticles that can be stably loaded with high concentrations of various antibiotics and engineered for burst or sustained release profiles. Aerosol-assisted evaporation-induced self-assembly enables modification of a nanoparticle surface with various targeting ligands and promotes effective uptake by infected cells cells.

Other aspects of embodiments of the present invention are directed to pharmaceutical compositions. Pharmaceutical compositions according to the present invention comprise a population of nanoparticles as otherwise described herein which may be the same or different and are formulated in combination with one or more pharmaceutically acceptable carrier, additive and/or excipient. The nanoparticles may be formulated alone or in combination with a bioactive agent (such as an antibiotic, an additional bioactive agent or an antiviral agent) depending upon the disease to be prevented and the route of administration (most often oral, as otherwise described herein). These compositions comprise nanoparticles as modified for a particular purpose (e.g. generating a therapeutic response, etc. Pharmaceutical compositions comprise an effective population of nanoparticles for a particular purpose and route of administration in combination with a pharmaceutically acceptable carrier, additive or excipient.

An embodiment of the present invention also relates to methods of utilizing the novel nanoparticles as described herein to generate an optional immunogenic response as well as to treat an infectious disease. Thus, in alternative embodiments, the present invention relates to a method of eliciting an immunogenic response in a host or patient (preferably, both a humoral and cell mediated response), treating, preventing and/or reducing the likelihood of disease in a subject or patient at risk for said disease, optionally treating a disease and/or condition comprising administering to a patient or subject in need an effective amount of a pharmaceutical composition as otherwise described herein.

The pharmaceutical compositions according to the present invention are particularly useful for administering an antibiotic in oral dosage form and treating an infectious disease, and/or preventing and/or reducing the likelihood of a number of disease states and/or conditions, especially diseases which are caused by microbes, such as bacteria, especially pathogenic/virulent bacteria.

As discussed in detail above, the porous nanoparticle core of the present invention can include porous nanoparticles having at least one dimension, for example, a width or a diameter of about 3,000 nm or less, about 1,000 nm or less, often about 500 nm or less, about 200 nm or less. Preferably, the nanoparticle core is spherical with a preferred diameter of about 500 nm or less, more preferably about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells according to the present invention are biocompatible. Antigens, drugs and other cargo components are often loaded by adsorption and/or capillary filling of the pores of the particle core up to approximately 50% by weight of the final protocell (containing all components). In certain embodiments according to the present invention, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In the present invention, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In preferred aspects of the invention, the lipid bilayer is fused onto the porous particle core to form the protocell. Protocells according to the present invention can include various lipids in various weight ratios, preferably including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

The lipid bilayer which is used to prepare protocells according to the present invention can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the protocell in order to improve the protocell colloidal stability.

In certain diagnostic embodiments, various dyes or fluorescent (reporter) molecules can be included in the protocell cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed protocell can then be observed by, for example, confocal fluorescence for use in diagnostic applications. In addition, as discussed herein, plasmid DNA can be used as cargo in protocells according to the present invention such that the plasmid may express one or more fluorescent proteins such as fluorescent green protein or fluorescent red protein which may be used in diagnostic applications.

In various embodiments, the protocell is used in a synergistic system where the lipid bilayer fusion or liposome fusion (i.e., on the porous particle core) is loaded and sealed with various cargo components with the pores (mesopores) of the particle core, thus creating a loaded protocell useful for cargo delivery across the cell membrane of the lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final protocell.

Pore surface chemistry of a nanoparticle material can be diverse. Attractive electrostatic interactions or hydrophobic interactions tend to control or enhance a loading capacity and a release rate. Higher surface areas can lead to higher loading of a cargo through these attractive interactions. In one embodiment, a porous nanoparticle core can be tuned in to be hydrophilic or progressively more hydrophobic and can be further treated to provide a more hydrophilic surface. For example, mesoporous silica particles can be further treated with ammonium hydroxide and hydrogen peroxide to provide a higher hydrophilicity.

Core particles dissolution rate may be varied or tuned by the degree of condensation of the particle. A fully condensed inorganic core structure (e.g., alum or silica) will dissolve in vivo at a slower rate than a less condensed structure.

A fusion and synergistic loading mechanism can be included for cargo delivery. For example, cargo can be loaded, encapsulated, or sealed, synergistically through liposome fusion on the porous particles. The cargo can include, for example, small molecule drugs (e.g. especially antibiotics), peptides, proteins, antibodies, DNA (especially plasmid DNA, including the preferred histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

As discussed above, electrostatics and pore size can play a role in cargo loading. For example, porous nanoparticles can carry a negative charge and the pore size can be adjusted from about 2 nm to about 10 nm or more. Negatively charged nanoparticles have a natural tendency to adsorb positively charged molecules and positively charged nanoparticles have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bilayer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, in a subject or patient. Due to the internalization of the cargo-loaded protocells in the target cell, cargo components can then be delivered into the target cells. In certain embodiments the cargo is an antigenic peptide or other small molecule, which can be delivered directly into the target cell for therapy. In other embodiments, negatively charged DNA or RNA (including shRNA or siRNA), especially including a DNA plasmid which is preferably formulated as histone-packaged supercoiled plasmid DNA preferably modified with a nuclear localization sequence can be directly delivered or internalized by the targeted cells. Thus, the DNA or RNA can be loaded first into a protocell and then into then through the target cells through the internalization of the loaded protocell.

Once bound and taken up by the target cells, the loaded protocells can release cargo components from the porous particle and transport the released cargo components into the target cell. For example, sealed within the protocell by the liposome fused bilayer on the porous particle core, the cargo components can be released from the pores of the lipid bilayer, transported across the protocell membrane of the lipid bilayer and delivered within the targeted cell. In embodiments according to the present invention, the release profile of cargo components in protocells can be more controllable as compared with when only using liposomes as known in the prior art. The cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the protocells can be pH-dependent.

In certain embodiments, the pH value for cargo is often less than 7, preferably about 4.5 to about 6.0, but can be about pH 14 or less. Lower pHs tend to facilitate the release of the cargo components significantly more than compared with high pHs, given that infected cells often have a lower pH than uninfected cells and because the endosomal compartments inside most cells are at low pHs (about 5.5); accordingly, the rate of delivery of cargo at the cell can be influenced by the pH of the cargo. Depending upon the cargo and the pH at which the cargo is released from the protocell, the release of cargo can be relative short (a few hours to a day or so) or a span for several days to about 20-30 days or longer. Thus, the present invention may accommodate immediate release and/or sustained release applications from the protocells themselves by providing release of cargo at lower or higher pH.

In certain embodiments, the inclusion of surfactants can be provided to rapidly rupture the lipid bilayer, thus facilitating transport of the cargo components across the lipid bilayer of the protocell, and in some cases, the targeted cell. In certain embodiments, the phospholipid bilayer of the protocells can be ruptured by the application/release of a surfactant such as sodium dodecyl sulfate (SDS), among others to facilitate a rapid release of cargo from the protocell into the targeted cell. In certain embodiments, the rupture of the lipid bilayer can in turn induce immediate and complete release of the cargo components from the pores of the particle core of the protocells. In this manner, the protocell platform can provide versatile delivery systems as compared with other delivery systems in the art. For example, when compared to delivery systems using nanoparticles only, the disclosed protocell platform can provide a simple system and can take advantage of the low toxicity and immunogenicity of liposomes or lipid bilayers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile.

In addition, the lipid bilayer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bilayer of the protocells can provide a fluidic interface (influenced by the lipids used and the amount of water in the lipid bilayer/multilayer) for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells, thus making the present invention far more therapeutical effective than prior art liposome compositions.

Alternatively, rather than immersing a particle core in a solution of the cargo, in another embodiment, the particle core may be assembled around the cargo. One way this may be accomplished is by combining precursors to the particle core with an antibiotic in solution and spray drying the combination. Representatively, for a particle core of mesoporous silica particle, the precursors may include hydrochloric acid (HCl), a surfactant such as catrimonium bromide (CTAB) and tetraethylorthosilade (TEOS) that may be combined with an antibiotic.

In one embodiment, (3-aminopropyl)triethoxysilane (APTES)-modified protocells may be utilized for random adsorption of cargo components. To promote adsorption, protocells can be soaked in a solution of the desired antibiotic(s) for 12 hours at 4° C. or longer and washed three times (e.g. with 1×PBS) to remove unencapsulated antibiotic. Protocells with a high degree of framework condensation will be used for random adsorption of antibiotic(s) since resulting particles will likely act as a depot and should, therefore, release antibiotic over time. Other aminosilanes such as (3-aminopropyl)-diethoxy-methylsilane (APDEMS), (3-aminopropyl)-dimethyl-ethoxysilane (APDMES) and (3-aminopropyl)-trimethoxysilane (APTMS) can be substituted for APTES, or a combination of aminosilanes can be used.

Once bound, the loaded protocells can release cargo components from the porous particle into the target cell or be taken up by the target cell wherein the cargo is released. For example, sealed within the protocell by the liposome fused bilayer on the porous particle core, the cargo components can be released from the pores of the lipid bilayer, transported across the protocell membrane of the lipid bilayer and delivered within the targeted cell or the nanoparticles can be taken up by the target cells wherein the cargo is released. In embodiments, the cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the protocells can be pH-dependent.

In addition to target release of cargo from protocells, in another embodiment, a systemic release is contemplated.

As discussed above, in certain embodiments, the pH value for cargo is often less than 7, preferably about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relatively short (a few hours to a day or so) or a span for several days to about 20-30 days or longer. Thus, the embodiments may accommodate immediate release and/or sustained release applications from the protocells themselves based upon the pH at which the cargo is released.

As discussed, in certain embodiments, the inclusion of surfactants preferably can be provided to rapidly rupture the lipid bilayer, transporting the cargo components across the lipid bilayer of the protocell as well as the targeted cell. In certain embodiments, the phospholipid bilayer of the protocells can be ruptured by the application/release of a surfactant such as sodium dodecyl sulfate (SDS), among others to facilitate a rapid release of cargo from the protocell into the targeted cell or systematically. In certain embodiments, the rupture of the lipid bilayer can in turn induce immediate and complete release of the cargo components from the pores of the particle core of the protocells. In this manner, the protocell platform can provide versatile delivery systems as compared with other delivery systems in the art. For example, when compared to delivery systems using nanoparticles only, the disclosed protocell platform can provide a simple system and can take advantage of the low toxicity and immunogenicity of liposomes or lipid bilayers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile.

In addition, the lipid bilayer and its fusion on porous particle core can be fine-tuned to control the loading, release, and targeting profiles and can further comprise fusogenic peptides and related peptides to facilitate delivery of the protocells for greater therapeutic effect.

Pharmaceutical compositions according to the present invention are preferably sterile and comprise an effective population of protocells as otherwise described herein formulated to effect an intended result (e.g. therapeutic result, immunogenic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions according to the present invention may also comprise an addition bioactive agent or drug, such as an antibiotic or antiviral agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities or more are employed. The composition may be administered to a subject by various routes, e.g. orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the invention include humans, companion animals, laboratory animals, and the like. The invention contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds according to the present invention may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the invention, with the remainder consisting essentially of suitable pharmaceutical excipients.

In preferred embodiments, a sterile, injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protoells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present invention.

Methods of treating patients or subjects in need for a particular disease state or infection comprise administration an effective amount of a pharmaceutical composition comprising therapeutic protocells and optionally at least one additional bioactive (e.g. antiviral) agent according to the present invention.

Diagnostic methods according to the present invention comprise administering to a patient in need an effective amount of a population of diagnostic protocells (e.g., protocells which comprise a target species, such as a targeting peptide which binds selectively to infected cells and a reporter component to indicate the binding of the protocells whereupon the selective binding of protocells to infected cells as evidenced by the reporter component (moiety) will enable a diagnosis of the existence of a disease state in the patient.

An alternative of the diagnostic method of the present invention can be used to monitor the therapy of a disease state in a patient, the method comprising administering an effective population of diagnostic protocells (e.g., protocells which comprise a target species, such as a targeting peptide which binds selectively to target cells and a reporter component to indicate the binding of the protocells to infected cells if the infected cells are present) to a patient or subject prior to treatment, determining the level of binding of diagnostic protocells to target cells in said patient and during and/or after therapy, determining the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated.

The invention is illustrated further in the following non-limiting examples.

### Example 1

### Protocell Design and Optimization

Supplementary Table 1 lists the MSNP and supported lipid bilayer (SLB) properties we can precisely control and how these properties can be used to tailor the in vitro and in vivo functionality of protocells. In the next three sections, we describe how we have applied these design rules to adapt protocells for high capacity loading and controlled release of various FDA-approved antibiotics.

SUPPLEMENTARY TABLE 1
Established fundamental design rules for the protocell platform. The MSNP and SLB properties that can
be precisely controlled to tailor various protocell parameters are listed, along with the resulting biological
effect(s) See Ashley, Carnes, Brinker, et al. Nature Materials (2011) for further details.

| MSNP or SLB Property | Protocell Parameter(s) | Biological Effect(s) |
|---|---|---|
| Size and Size Distribution | Biodistribution, internalization efficiency | Tailor the concentration of drug(s) in specific |
| | | organs, tissues and/or cells |
| MSNP Charge | MSNP-SLB interaction | Balance extracellular drug retention and intracellular |
| | | drug release by optimizing SLB stability |
| Pore Size | Loading capacity, type(s) of cargo | Reduce dose by decreasing the number of |
| | molecules that can be loaded, release rates, | nanoparticles that have to reach target site(s) in |
| | SLB fluidity | order to see an effect and/or by delivering drug |
| | | cocktails |
| Pore Chemistry | Loading capacity, type(s) of cargo | Same as above |
| | molecules that can be loaded | |
| Degree of Silica Framework | Release rates, biodegradability | Reduce the frequency and duration of treatment |
| Condensation | | through optimized release profiles; enhance |
| | | biocompatibility by ensuring nanoparticles and/or |
| | | byproducts are benignly excreted |
| SLB Charge | Non-specific (vs. specific) uptake | Maximize the concentration of drug(s) in target |
| | | site(s) by decreasing non-specific interactions |
| SLB Fluidity | Mobility of targeting ligands, specific | Maximize the concentration of drug(s) in target |
| | binding affinities | site(s) by increasing specific interactions |
| Thickness of Lipid Coating, | Tailorable release rates under various | Balance extracellular drug retention and |
| Presence/Number of Intra- or | intracellular conditions | intraceullular drug release by optimizing SLB |
| Interbilayer Bonds | | stability |
| Degree of PEGylation | SLB stability, colloidal stability | Maximize the concentration of drug(s) in target |
| | | site(s) by minimizing unwanted cargo release; |
| | | enhance biocompatibility by minimizing serum- |
| | | induced aggregation |
| Type and Density of | Specific binding and uptake | Maximize the concentration of drug(s) in target |
| Targeting Ligand(s) on SLB | | cell(s) to decrease dose and minimize off-target |
| Surface | | effects |
| Incorporation of | Cytosolic cargo delivery | Tailor the concentration of drug(s) in specific |
| Endo/Lyso/Phagosomolytic | | intracellular locations |
| Peptides on the SLB | | |

In the aerosol-assisted EISA process (see Supplementary FIG. 1), a dilute solution of a metal salt or metal alkoxide is dissolved in an alcohol/water solvent along with an amphiphilic structure-directing surfactant or block co-polymer; the resulting solution is then aerosolized with a carrier gas and introduced into a laminar flow reactor. Solvent evaporation drives a radially-directed self-assembly process to form particles with systematically variable pores sizes (2 to 50-nm), pore geometries (hexagonal, cubic, lamellar, cellular, etc.), and surface areas (100 to >1200 m2/g). Aerosol-assisted EISA, additionally, produces particles compatible with a variety of post-synthesis processing procedures, enabling the hydrodynamic size to be varied from 20-nm to >10-μm and the pore walls to be modified with a wide range of functional moieties that facilitate high capacity loading of physicochemically disparate diagnostic and/or therapeutic molecules. Importantly, aerosol-assisted EISA produces MSNPs that can be easily dispersed in a variety of aqueous and organic solvents without any appreciable aggregation, which enables us to load drugs that have high and low solubility in water. Our MSNPs are also easily encapsulated within anionic, cationic, and electrically-neutral SLBs via simple liposome fusion. In contrast, MSNPs generated using solution-based techniques tend to aggregate when the pH or ionic strength of their suspension media changes,10 typically require complex strategies involving toxic solvents to form. SLBs,11, 12 and have maximum loading capacities of 1-5 wt %,13 which, as described in the next section, our MSNPs exceed by an order of magnitude.
MSNPs with Reproducible Properties can be Synthesized in a Scalable Fashion Via Aerosol-Assisted Evaporation-Induced Self-Assembly.

MSNPs formed via aerosol-assisted EISA have an extremely high surface area (>1200 m2/g), which enables high concentrations of various therapeutic and diagnostic agents to be adsorbed within the core by simple immersion in a solution of the cargo(s) of interest. Furthermore, since aerosol-assisted EISA yields MSNPs that are compatible with a range of post-synthesis modifications, the naturally negatively-charged pore walls can be modified with a variety of functional moieties, enabling facile encapsulation of physicochemically disparate molecules, including acidic, basic, and hydrophobic drugs, proteins, small interfering RNA, minicircle DNA vectors, plasmids, and diagnostic agents like quantum dots and iron oxide nanoparticles.1-3 As demonstrated by FIG. 1A, MSNPs formed via aerosol-assisted EISA have loading capacities of 20-55 wt % for various individual antibiotics and 10-15 wt % for individual antibiotics in three-drug-cocktails; it is important to note that these capacities are 13 and 15-17 We are able to achieve high loading capacities for acidic, basic, 2 As shown in FIG. 1B, MSNPs with a low degree of silica condensation release 100% of encapsulated levofloxacin within 12 hours, while MSNPs with a high degree of silica condensation release encapsulated levofloxacin over a period of 2 weeks (see Supplementary FIG. 2B); it is important to note that these data represent burst and sustained release.

FIG. 1, panel A shows the loading capacities of MSNPs with 2.5-nm pores for several physicochemically disparate antibiotics. Approximate weight percentages of individual antibiotics when MSNPs are loaded with cocktails of levofloxacin (LEV), doxycycline (DOX), and gentamicin (GEN) or ceftazidime (CEF), sulfamethoxazole (SMX), and trimethoprim (TMP) are included at the far right. FIG. 1, panel B shows time-dependent release of levofloxacin from MSNPs with a low or high degree of silica (SiO2) framework condensation upon incubation in a simulated body fluid (10% serum, pH 7.4) at 37° C. A low degree of silica condensation was achieved using acidified ethanol to extract structure-directing surfactants, while a high degree of silica condensation was promoted via thermal calcination. FIG. 1, panel C shows mean fluorescence intensities of THP-1, A549, and HepG2 after incubation with DOPC protocells labeled with pHrodo Red and modified with 5 wt % of human Fcγ, 5 wt % of human complement C3, 30 wt % of mannosylated cholesterol, 5 wt % of human ephrin B2, or 5 wt % of SP94. Protocells coated with just DOPC (electrically neutral) or with a cationic lipid (DOTAP) were included as controls. FIG. 1, panel D shows the number of colony-forming units (CFUs) of Ft LVS that remain upon treatment of LVS-infected THP-1 or A549 cells with empty protocells, free levofloxacin (Levo), or levofloxacin loaded in DOPC protocells or DPPC liposomes modified with 5 wt % of Fcγ and H5WYG. Error bars=mean±std. dev. for n=3.

Optimization of Pore Chemistry Enables High Capacity Loading of Physicochemically Disparate Antibiotics and Antibiotic Cocktails, while Optimization of Silica Framework Condensation Results in Tailorable Release Rates.

Despite recent improvements in encapsulation efficiencies and serum stabilities, state-of-the-art liposomes, multilamellar vesicles, and polymeric nanoparticles still suffer from several limitations, including complex processing techniques that are highly sensitive to pH, temperature, ionic strength, presence of organic solvents, lipid or polymer size and composition, and physicochemical properties of the cargo molecule, all of which impact the resulting nanoparticle's size, stability, entrapment efficiency, and release rate. 10-fold higher than other MSNP-based delivery vehicles 100 to 1000-fold higher than similarly-sized liposomes and polymeric nanoparticles and hydrophobic drugs by modulating the pore chemistry (see Supplementary FIG. 2A) and by altering the solvent used to dissolve the drug prior to loading. Unlike MSNPs formed using solution-based techniques, MSNPs formed via aerosol-assisted EISA are compatible with all aqueous and organic solvents, which ensures that the maximum concentration of drug loaded within the pore network is essentially equivalent to the drug's maximum solubility in its ideal solvent. Another unique feature of our MSNPs is that the rate at which encapsulated drug is released can be precisely modulated by varying the degree of silica framework condensation and, therefore, the rate of its dissolution via hydrolysis under physiological conditions.

Our ability to achieve high loading capacities for individual antibiotics and antibiotic cocktails should enable us to develop a protocell formulation that will reduce the required dose of levofloxacin compared to free drug. Furthermore, our ability to precisely tailor release rates will allow us to control the pharmacokinetics of protocell-encapsulated levofloxacin over a wider range than is achievable with free antibiotics or antibiotic-loaded liposomes be further tailored between these extremes. Our ability to achieve high loading capacities for individual antibiotics and antibiotic cocktails should enable us to develop a protocell formulation that will reduce the required dose of levofloxacin compared to free drug. Furthermore, our ability to precisely tailor release rates will allow us to control the pharmacokinetics of protocell-encapsulated levofloxacin over a wider range than is achievable with free antibiotics or antibiotic-loaded liposomes.

In contrast, we have shown that lipid bilayers supported on mesoporous silica particles (see TEM image in FIG. 2) have a high degree of stability in neutral-pH buffers and serum-containing simulated body fluids, regardless of the charge or fluidity of lipids used to form the SLB.² In addition to being highly stable, the SLB provides a biocompatible interface with tailorable fluidity for display of functional molecules, such as polyethylene glycol (PEG) and targeting ligands.² We have demonstrated that protocells with SLBs composed of the zwitterionic, fluid lipid, 1,2-dioleoyl-sn-glycerol-3-phosphocholine (DOPC) with 30 wt % cholesterol and 10 wt % of PEG have a high degree of colloidal stability (see Supplementary FIG. 3A) and stably encapsulate small molecule drugs, like levofloxacin for up to 4 weeks (see Supplementary FIG. 3B) when incubated in a serum-containing simulated body fluid at 37° C. In dramatic contrast, MSNPs coated with cationic polymers, such as polyethyleneimine (PEI) rapidly aggregate in the presence of serum (see Supplementary FIG. 3A), and levofloxacin-loaded liposomes rapidly leak their encapsulated drug (see Supplementary FIG. 3B), even when composed of the fully-saturated lipid, 1,2-distearoyl-sn-glycero-3-phosphocholine (DSPC), which has a higher packing density than fluid lipids (e.g. DOPC) and should limit diffusion of drug across the bilayer.² Although protocells are highly stable under neutral pH conditions, the SLB can be destabilized under endo/lyso/phagosomal conditions, such as acidic pH (see Supplementary FIG. 3C); SLB destabilization, as described in the next section, triggers dissolution of the MSNP core and enables intracellular delivery of encapsulated drugs.^{1-3, 18} Interbilayer-crosslinked SLMs, produced through inclusion of 1,2-dioleoyl-sn-glycero-3-phosphoethanolamine-N-[4-(p-maleimidophenyl)butyramide] (18:1 MPB PE) in the liposome formulation, followed by use of dithiothreitol to crosslink maleimide-containing headgroups of opposed lipid layers,18 have the highest degree of stability and can only be degraded in environments that mimic phagolysosomes (see Supplementary FIG. 3C).

Fusion of Liposomes to Drug-Loaded MSNPs Creates a Coherent SLB that Enhances Colloidal Stability and Enables Long-Term Cargo Retention.

Liposomes and multilamellar vesicles have poor intrinsic chemical stability, especially in the presence of serum, which decreases the effective concentration of drug that reaches target cells and increases the potential for systemic toxicity. It is important to note, however, that the stability of the SLB, which influences the rate at which protocells release drug under intracellular conditions by initiating MSNP dissolution, can be tailored by optimizing the thickness and degree of crosslinking in the lipid bi/multilayer. For example, inclusion of photopolymerizable lipids, such as 1-palmitoyl-2-(10,12-tricosadiynoyl)-sn-glycero-3-phosphocholine (16:0-23:2 Diyne PC), enables formation of an intrabilayer-crosslinked SLB. If a higher degree of stability is required, supported lipid multilayers (SLMs) can be formed around protocells via liposome fusion in the presence of divalent cations. Our ability to control the thickness of the lipid layer, as well as the approximate number of intra- and/or interbilayer bonds, will be critical to balancing the stability of orally-administered protocells with the rates of extra- and intracellular antibiotics release necessary to treat respiratory tularemia.

A number of factors govern cellular uptake and processing of nanoparticles, including their size, shape, surface charge, and degree of hydrophobicity. Additionally, a variety of molecules, including peptides, proteins, aptamers, and antibodies, can be employed to trigger active uptake by a plethora of specific cells. We have previously shown that incorporation of targeting and endosomolytic peptides that trigger endocytosis and endosomal escape on the protocell SLB enables cell-specific delivery and cytosolic dispersion of encapsulated cargoes. As importantly, we have shown that Modifying the SLB with Various Targeting Ligands Promotes Efficient Uptake of Levofloxacin-Loaded Protocells by Model Host Cells and Enables Highly Efficacious Killing of Intracellular F. tularensis. In order to effectively kill intracellular F. tularensis and B. pseudomallei, nanoparticle delivery vehicles must release antibiotics directly into the cytosol of host cells, which not only increases the concentration of drug in the vicinity of the pathogen but is also important since many classes of antibiotics, including β-lactams, lincosamides, and fluoroquinolones, show poor penetration or rapid efflux from mammalian cells. SLB fluidity can be tuned to enable exquisite (sub-nanomolar) specific affinities for target cells at extremely low targeting ligand densities (˜6 targeting peptides per protocell) and that SLB charge and degree of PEGylation can be modulated to reduce non-specific interactions, resulting in protocells that are internalized by target cells 10,000-times more efficiently than non-target cells.

As shown by FIG. 1D, cell-specific internalization of Fcγ-targeted protocells by THP-1 cells enables cell-specific delivery of levofloxacin and effective killing of intracellular F. tularensis, subspecies holarctica live vaccine strain (LVS), when MSNP cores exhibiting burst release kinetics are used and the SLB is further modified with a high density of the endo/lyso/phagosomolytic peptide, H5WYG,26 which disrupts endo/lyso/phagosomal membranes upon vesicle acidification and triggers dispersion of MSNP cores in the cytosol. Furthermore, with only 2 wt % loading of levofloxacin (˜1/20th of the protocell's maximum capacity), cytotoxicity toward intracellular LVS exceeded that of free levofloxacin and levofloxacin-loaded DSPC liposomes. We have also shown that levofloxacin-loaded protocells are not toxic to host cells (see Supplementary FIG. 4B), even upon burst release of 1 mg/mL (˜25,000 times the MIC90 value of levofloxacin).

In summary, protocells are highly flexible and modular. In dramatic contrast to other state-of-the-art nanoparticle delivery vehicles, we can load high concentrations of physicochemically disparate molecules and specifically tailor release rates without altering the protocell's size, size distribution, stability, or synthesis strategy. We can, furthermore, modulate properties of the SLB and MSNP core entirely independently, which enables us to optimize such properties as surface charge, colloidal stability, and targeting specificity independently from overall size, type of cargo(s), loading capacity, and rate of release and offers us several options for engineering and tailoring triggered cargo release.^{27,28} compared to solid silica nanoparticles, mesoporous silica nanoparticles exhibit reduced toxicity and hemolytic activity since their surface porosity decreases the contact area between surface silanol moieties and cell membranes;^{7, 29, 30} (4) the high internal surface area (>1000 m2/g) and ultra-thinness of the pore walls (<2-nm) enable MSNPs to dissolve, and soluble silica (e.g. silicic acid, Si(OH)4) has extremely low toxicity;28, 31 and (5) in the case of protocells, the SLB further reduces interactions between surface silanol moieties and cell membranes and confers immunological behavior comparable to liposomes.² To confirm these predictions, we have evaluated the biocompatibility and biodegradability of protocells after repeat intravenous (i.v.) or intramuscular (i.m.) injections in Balb/c and C57Bl/6 mice. Balb/c mice injected i.v. or i.m. with 200 mg/kg doses of PEGylated protocells three times each week for 3 weeks showed no signs of gross or histopathological toxicity; given their high loading capacity, this result indicates that protocells can deliver at least 900 mg/kg (nearly 10 times the recommended dosing schedule for oral Levaquin) of small molecule drugs with either burst or sustained release kinetics. Furthermore, our collaborators at the UCLA Center for Environmental Implications of Nanotechnology (CEIN) have shown that MSNPs are biodegradable and are ultimately excreted in the urine and feces as silicic acid.⁵ Finally, we have shown that protocells modified with high densities (up to 10 wt %) of small molecules (e.g. peptides, folate).

Protocells are Biocompatible, Biodegradable, and Non-Immunogenic.

Several reasons support the observation that mesoporous silica nanoparticles have low toxicity profiles in vivo: (1) silica is accepted as ‘Generally Recognized As Safe’ (GRAS) by the U.S. FDA; (2) recently, solid, dye-doped silica nanoparticles received approval from the FDA for targeted molecular imaging of cancer; induce neither IgG nor IgM responses when injected in C57Bl/6 mice at a total dose of 400 mg/kg.

The Biodistribution of Protocells can be Controlled by Tuning their Hydrodynamic Size and Size Distribution.

Since liposomes and multilamellar vesicles are the most similar nanoparticle delivery vehicles to protocells, we make every effort to benchmark the performance of protocells against the performance of lipid-based nanoparticles. We and others have found that liposomes and multilamellar vesicles, despite being more elastic that protocells, have biodistribution that are largely governed by their overall size and size distributions, an observation that holds true for protocells as well (see below) and subject to slight variations in lipid content, buffer pH and ionic strength and the chemical properties of the cargo molecules.

In addition to proving that protocells are highly biocompatible, we have also shown that³⁵⁻³⁹ they can be engineered for both broad distribution and persistence within target tissues. Tuning particle size and size distribution offers the most direct mechanism for altering bulk biodistribution. We have found that intermediate-sized protocells (150±11 nm, 250±17 nm) start to accumulate in the liver, spleen, and kidneys within 48 hours of i.v. or i.m. administration (see Supplementary FIG. 5B), while larger protocells (>300-nm) accumulate in the lungs with 2-4 hours, observations we have confirmed using ex vivo imaging of organs. Conversely, smaller protocells (25±4 nm, 50±7 nm) rapidly accumulate in the kidney and bladder within the first 48 hours of i.v. or i.m. injection (see Supplementary FIG. 5B) but can also be observed at high concentration in the blood for more than 10 days (see Supplementary FIG. 5C) and are capable of penetrating deep within a variety of tissues (e.g. the red and white pulp of the spleen. We have also monitored the state of protocells in the blood and urine and have found that protocells do not aggregate in the blood (see Supplementary FIG. 6A-C) but appear in the urine in various states of dissolution and aggregation as early as 1-hour post-injection (see Supplementary FIG. 6D). These observations suggest that we can find an optimum combination of sizes that will simultaneously maximize passive accumulation of protocells within desired organs, as well as broad distribution to allow for sustained, systemic drug release and for further targeting of specific cells and tissues. We will apply our knowledge of how protocells distribute once they are in circulation to optimizing them for oral delivery. We are confident that the protocell's flexible, modular nature will enable us to achieve significant intestinal penetration; based on published reports of orally-administered solid lipid and polymeric nanoparticles, we expect that once protocells cross the intestinal epithelium, they should enter circulation within 1-2 hours.

### Example 2

### Orally-Administered Antibiotic Protocells for the Treatment of Respiratory Tularemia

Here we describe the disease progression of respiratory tularemia, which has guided our design of the three protocell formulations depicted in FIG. 2, and provide a thorough justification for our choice of oral administration over inhalation-based delivery modalities. We then provide tables that either describe features of the three protocell formulations or supply a detailed list of candidate enzymes and their cognate substrates, which we will use to develop infection-triggered release strategies. We also provide a series of schematics that are intended to illustrate how we expect orally-administered protocells to distribute as a function of time after intestinal penetration, how we expected Fcγ-targeted protocells with substrate-capped MSNP pores to interact with and selectively release encapsulated drug(s) within Ft-infected cells, and how we will construct MSNPs with substrate-capped pores, generate tethered SLBs that fully encapsulate substrate-modified MSNPs, and conjugate the SLBs with a number of different functional molecules, including PEG, CD47, Fcγ, and the RGD and H5WYG peptides. Finally, we present images of our novel ex ovo avian embryo model that demonstrate its range of capabilities and provide details about the types of biological measurements we will make during our proposed Fischer 344 rat and Cynomolgus macaque safety studies.

Disease Progression of Respiratory Tularemia.

Tularemia in humans is most often a zoonotic disease acquired through the bite of an arthropod vector, which typically presents as a necrotic lesion at the site of infection, as well as draining lymphadenopathy and high fever. Systemic disease can be caused by cutaneous, respiratory, or gastrointestinal transmission, but the most severe symptoms follow respiratory exposure.¹ Furthermore, inhalation of live F. tularensis (Ft) is the most likely route of transmission in the event of a bioterrorism attack,¹ leading us to focus our efforts primarily on the treatment of respiratory tularemia. Primary pneumonic tularemia caused by Ft ssp. tularensis (also known as type A) has been reported to have mortality rates as high as 60% in untreated patients, whereas disease caused by Ft ssp. holarctica (also known as type B) strains is usually self-limiting;^{2, 3} for these reasons, we will employ the Ft type A strain, SCHU S4, in all studies focused on treatment of respiratory tularemia. Aerosol challenge of Rhesus macaques with Ft SCHU S4 has indicated that early bronchiolitis develops within 24 hours of infection and progresses to bronchopneumonia within 72-96 hours, while lymphadenitis, splenitis, and hepatitis develop at 24-72 hours post-infection.1 Another study showed that SCHU S4 renders African green monkeys moribund within 7-11 days of aerosol infection and causes necrotic inflammatory loci in the lungs, liver, spleen, and lymph nodes.⁴ Importantly, respiratory tularemia is thought to follow a similar progression in humans,⁵ necessitating systemic distribution of antibiotics, as well as delivery of antibiotics to specific organs, including the lungs, liver, spleen, and lymph nodes. On a cellular level, Ft is known to infect macrophages, dendritic cells, neutrophils, hepatocytes, and lung epithelial cells. It is internalized via phagocytosis, but can rapidly escape the phagosome and replicate in the cytosol of the host cell. Once in the cytosol, Ft is recognized by innate immune defenses including the inflammasome, which can trigger caspase-1-mediated pyroptosis of the infected cell, as well as release of pro-inflammatory cytokines, including IL-1β and IL-18.1,⁶ In the latter stages of infection, Ft-infected cells undergo caspase-3-dependent apoptosis,¹ and Ft released from apoptotic cells can infect neighboring cells or spread systemically.

Advantages of Oral Administration.

The use of nanoparticles for treatment of tularemia has thus far been limited to untargeted liposomes loaded with ciprofloxacin, a fluoroquinolone antibiotic similar to levofloxacin.^{7, 8} While this approach proved efficacious when 30 μg of liposomal ciprofloxacin was administered as an aerosol either 24 hours before or 72 hours after infection, the 20-minute exposure time that was required for inhalation-based administration to deliver a sufficient concentration of drug to target sites renders this approach sub-optimal.⁸ These studies did confirm, however, the potential for nanoparticles to successfully widen the narrow window during which antibiotics must be administered to ensure a favorable outcome. In addition to the long exposure times required for inhalation-based delivery to be effective, dry powder inhalers, metered-dose inhalers, and nebulizers have relatively low adsorption efficiency, with only ˜1% of inhaled nanoparticles ending up in circulation.^{9, 10}

Based on this approximation and other literature reports, we calculated that no more than 1,011 protocells (˜250 μg) can be inhaled in a single breath; assuming a loading capacity of 40 wt %, this means that protocells should be able to deliver ˜1 μg of drug to the bloodstream, which exceeds the concentration of drug that can be delivered to the bloodstream via aerosolized liposomes (0.3 μg) but is 40-5,000 times less than our estimates for oral protocell administration (see below). Other limitations of inhalation-based delivery include that both the droplet size and nanoparticle size must be precisely optimized to ensure adequate lung penetration and deposition of nanoparticles.¹⁰ Finally, the dose administered to animals is inherently difficult to control and measure for inhalation-based techniques, including aerosolization, as well as intranasal and intratracheal administration. For all of the reasons described above, we ultimately decided to pursue oral administration of protocells in this proposal.

Researchers have been pursuing gastrointestinal delivery of therapeutic peptides, proteins, and small molecules using nano- and microparticles since the late 1980s, and several comprehensive review articles are available that describe how nanoparticle size, charge, and surface modifications can be optimized to maximize stability in the gastrointestinal tract, penetration through mucosal barriers, translocation through the intestinal epithelium (by paracellular transport, passive or receptor-mediated transcytosis across epithelial cells, and/or transport via M cells in Peyer's patches), and dispersion in the lymphatic and/or circulatory systems.¹¹⁻¹³ Although orally-administered nanoparticles must be able to survive the acidic, proteolytic environment of the stomach and effectively penetrate mucosal and cellular barriers in the small or large intestine, the general consensus is that 2-10% of orally-administered nanoparticles end up in circulation.¹¹⁻¹⁵ Based on Institutional Animal Care and Use Committee regulations, we can administer 20 mL/kg of a liquid slurry of protocells (25 mg/mL at saturation) to rats; assuming an average weight of ˜500 g, a loading capacity of 40 wt % (41.6 wt % for levofloxacin), and that 5% of adsorbed protocells end up in circulation, we estimate that protocells can deliver a minimum of 5 mg of drug into the bloodstream with a single oral dose. Furthermore, even if 100% of orally-administered protocells were absorbed by the intestine and ended up in circulation, the total administered dose of levofloxacin (˜200 mg/kg) would remain a fraction of the oral LD50 for levofloxacin in rats (˜1500 mg/kg).

Since direct oral administration will necessitate that protocells are able to survive gastric conditions, coating protocells in an enterically-coated capsule could be used to facilitate oral administration. Assuming a capsule volume of 0.08 mL for rats and a loading capacity of 40 wt %, we expect to be able to administer approximately 800 μg of drug per capsule. Therefore, assuming that 5% of administered particles end up in circulation, we expect that protocells should be able to deliver 40 μg of drug systemically. Based on prior reports that a total aerosolized dose of 30 μg of liposomal ciprofloxacin (˜300 ng of which ended up in circulation) confers protection to mice when administered 24 hours in advance and 72 hours after lethal challenge with 10 times the LD50 for Ft,¹⁸ we expect that a single capsule should be sufficient to treat respiratory tularemia when administered pre-symptomatically or after exposure but prior to onset of symptoms. Furthermore, by using a combination of particles with release rates ranging from 2000 ng/day (nearly 10 times the total systemic concentration achieved using liposomal ciprofloxacin) to ˜25 μg/day, we should be able to achieve high initial systemic drug concentrations, as well as sustained systemic drug concentrations for nearly two weeks. If the capsule capacity proves insufficient, however, we will optimize the thickness and degree of crosslinking in the protocell's lipid shell to enable direct oral administration.

SUPPLEMENTARY TABLE 2
MSNP and SLB properties that will be modulated to enable rapid, triggerable release of a high concentration
of levofloxacin in Ft-Infected cells, as well as sustained concentrations of levofloxacin in the blood
and organs known to be infected by Ft (lungs, liver, spleen, and lymph nodes). Further details about the
synthesis and characterization of these protocell formulations are in the task 4.3 and 4.4 descriptions:
percentages in the ‘Anticipated SLB Composition’ column refer to weight percent, and cholesterol
is abbreciated ‘CH.’. MSNP pores will be ‘capped’ with substrate(s) of enzymes activated in
response to Ft infection; we will use lipid tethers (see Supplementary FIGS. 12-13) to space the SLBs
of these protocells away from the MSNP core in order to accommodate the substrate molecules.

| | | | | | | SLB | Anticipated |
|---|---|---|---|---|---|---|---|
| | | MSNP | Target | Substrate - | Anticipated | Tethered | Surface |
| Protocell | | Size | Release | Capped | SLB | to MSNP | Molecule |
| Formulation | Function | (nm) | Rate | Pores? | Composition | Surface? | Densities |
| i | Systemically circulate | <100 | 2 μg/day | No | 19% DSPC, | No | 10% PEG- |
| | and release drug for >10 | | for 10-14 | | 30% CH, 30% | | 2000, 10% |
| | days | | days | | 16:0-23:2 | | RGD, 1% |
| | | | | | Diyne PC, 21% | | CD47 |
| | | | | | DSPE | | |
| ii | Become internalized by | 100-600 | 2 μg/day | No | 15% DSPC, | No | 10% PEG- |
| | potential host cells and/or | | for 10-14 | | 30% CH., 30% | | 2000, 10% |
| | concentrated in | | days | | 16:0-23:2 | | RGD, 5% |
| | potentially effected | | | | Diyne PC, 25% | | Fcγ |
| | organs (lungs, liver, | | | | DSPE | | |
| | spleen, lymph nodes) and | | | | | | |
| | release drug for >10 days | | | | | | |
| iii | Become internalized by | 100-600 | 10-15 μg | Yes | 10% DSPC | Yes | 10% PEG- |
| | potential host cells and | | in <12 hours | | 30% Ch., 30% | | 2000, 10% |
| | rapidly release drug upon | | | | 16:0-23:2 | | RGD, 5% |
| | Ft infection | | | | Diyne PC, 17- | | Fcγ, 5% |
| | | | | | 30% DSPE | | H5WYG |

SUPPLEMENTARY TABLE 3
Candidate enzyme/substrate pairs to drive infection-triggered release of antibiotics
from protocells at sites of Ft infection or Ft-elicited inflammation.

| Location of Enzyme | Host Enzyme | Substrates | References |
|---|---|---|---|
| Intracellular | Caspase-1 | pro-IL1β, pro-IL18, pro-IL33 | 16-19 |
| Intracellular | Caspases 3, 7 | PARP-1, vimentin, α-tubulin | 16-19 |
| Intra/Extracellular | Granzymes | pro-IL1β, PARP-1, vimentin, α-tubulin, fibronectin, | 20-26 |
| | | ApeI, ICAD | |
| Intra/Extracellular | Neutrophil Elastase, | pro-IL1β, SP-A, pro-chemerin | 27-31 |
| | Proteinase 3 | | |
| Intra/Extracellular | Cathepsin G | SP-A, pro-chemerin, fibronectin | 29, 30, 32 |
| Intra/Extracellular | Mast Cell Chymase | pro-IL1β, pro-chemerin, fibronectin | 27, 29, 32 |
| Extracellular | Matrix Metalloproteinases | fibronectin, collagens, PG core protein, pro-IL8 | 31, 33, 34 |

SUPPLEMENTARY TABLE 4
Routine biological measurements that will be made during Fisher 344 rat (task 4.12) and Cynomolgus
macaques (task 4.13) safety studies. ‘Exposure’ refers to oral dosing of levofloxacin-
loaded protocells. ‘Toxicokinetics’ refers to the pharmacokinetics of any observed toxicity.

| Biological Measurement | Fischer 344 Rat | Cynomolgus macaques | Measurement Frequency |
|---|---|---|---|
| General Clinical Observation | X | X | Daily |
| Detailed Physicals | | X | Prior to exposure, subsequent to |
| | | | exposure, and at recovery |
| Detailed Clinical Observations | X | X | Twice weekly during exposure and |
| | | | recovery |
| Ophthalmology | X | X | Prior to exposure, subsequent to |
| | | | exposure, and at recovery |
| EKG Readings (GTc) | | X | Prior to exposure, subsequent to |
| | | | exposure, and at recovery |
| Pulmonary Function Testing | X | X | Not always required but preferred |
| Body Weight | X | X | Twice weekly during exposure and |
| | | | recovery |
| Blood Sampling | X | X | After first and last exposure, satellite |
| | | | animals for rat study |
| Hematology, Chemistry, | X | X | At end of exposure and recovery; |
| Urinalysis | | | terminal for rats |
| Gross Necropsy | X | X | Terminal |
| Organ Weight | X | X | Terminal |
| General Histopathology | X | X | Terminal |
| Toxicokinetics | X | X | After first and last exposure; might |
| | | | be conducted outside of general |
| | | | toxicity assessments |

### REFERENCES

1. Parmely M J, et al. FEMS Microbiology Letters 301: 1-11 (2009).
2. Dennis Dt ITVHDA, et al. JAMA 285: 2763-73 (2001).
3. SjÖStedt A. Annals of the New York Academy of Sciences 1105: 1-29 (2007).
4. Twenhafel N, et al. Vet Pathol 46: 698-706 (2009).
5. Lamps L W, et al. Mod Pathol 17: 489-95 (2004).
6. Henry T, et al. Cellular Microbiology 9: 2543-51 (2007).
7. Conley J, et al. Antimicrobial Agents and Chemotherapy 41: 1288-92 (1997).
8. Wong J P, et al. Journal of Controlled Release 92: 265-73 (2003).
9. Buzea C, et al. Biointerphases 2: MR17-MR71 (2007).
10. Geiser M, et al. Particle and Fibre Toxicology 7: 2 (2010).
11. Florence A. Pharmaceutical Research 14: 259-66 (1997).
12. Florence A T. Drug Discovery Today: Technologies 2: 75-81 (2005).
13. Woitiski C, et al. BioDrugs 22: 223-37 (2008).
14. Dang Y-J, et al. Chinese Medicine 8: 1 (2013).
15. He C, et al. Biomaterials 33: 8569-78 (2012),
16. Luthi A U, et al. Cell Death Differ 14: 641-50 (2007).
17. Walsh J G, et al. Proceedings of the National Academy of Sciences 105: 12815-9 (2008).
18. Walsh J G, et al. Journal of Biological Chemistry 286: 32513-24 (2011).
20. Bovenschen N, et al. Journal of Biological Chemistry 284: 3504-12 (2009).
21. Chowdhury D, et al. Annual Review of Immunology 26: 389-420 (2008).
22. de Poot SAH, et al. Biochemical Journal 437: 431-42 (2011).
23. Guo Y, et al. The Journal of Immunology 185: 5348-59 (2010).
24. Hua G, et al. The Journal of Immunology 182: 2152-9 (2009).
25. van Domselaar R, et al. Reviews in Medical Virology 21: 301-14 (2011).
26. Zhao T, et al. Journal of Biological Chemistry 282: 12104-11 (2007).
27. Afonina Inna S, et al. Molecular Cell 44: 265-78 (2011).
28. Croker B A, et al. Towards a Four-Dimensional View of Neutrophils #. In T Leucocytes, pp. 87-99
29. Guillabert A, et al. Journal of Leukocyte Biology 84: 1530-8 (2008).
30. Moraes T J, et al. Current Opinion in Hematology 13: 21-7 (2006).
31. Taggart C C, et al. American Journal of Respiratory and Critical Care Medicine 171: 1070-6 (2005).
32. Korkmaz B, et al. FEBS Journal 278: 2635-46 (2011).
33. Birkedal-Hansen H, et al. Critical Reviews in Oral Biology & Medicine 4: 197-250 (1993).
34. Imai K, et al. Biochem. J 322: 809-14 (1997).
35. Kruse C G, et al. Recueil des Travaux Chimiques des Pays-Bas 98: 371-80 (1979).

What is claimed is:

1. An antibiotic protocell comprising:
(a) mesoporous silica nanoparticle comprising about 10% to about 70% by weight of one or more antibiotics and having a pore size of approximately 1 nm to approximately 75 nm, a surface area of approximately 75 m²/g to approximately 1,500 m²/g and a hydrodynamic diameter of approximately 50 nm to approximately 50 μm; and
(b) a lipid bilayer or multilayer bi- or multilayer which encapsulates the nanoparticle and which optionally comprises (1) an optionally-thiolated PEG and/or (2) at least one targeting ligand which is conjugated to the outer surface of the lipid bi- or multilayer and which is specific for binding one or more receptors of a bacterially-infected host cell.

2. The protocell according claim 1 wherein said lipid bilayer comprises at least one PEGylated lipid in combination with at least one non-pegylated lipid.

3. The protocell according to claim 1 or 2 wherein said lipid bilayer further comprises PEGylated cholesterol.

4. The protocell according to claim 2 or 3 wherein said PEGylated lipid comprises about 0.0001% to about 25% by weight of the lipid bi- or multilayer.

5. The protocell of any of claims 1-4 wherein the mesoporous silica nanoparticle is made by an aerosol-assisted evaporation-induced self-assembly process, and wherein the charge and/or hydrophobicity of the mesoporous silica nanoparticle are optionally varied by the addition of one or more aminosilanes and/or trimethylsilyl group capping agents depending upon the charge and/or hydrophobicity of the one or more antibiotics, and wherein the maximum concentration of antibiotic loaded within the nanoparticle's pore network is approximately equal to the antibiotic's maximum solubility in its ideal solvent.

6. The protocell of claim 5, wherein:
(a) the aminosilanes are selected from the group consisting of (3-aminopropyl)triethoxysilane (APTES), p-aminophenyltrimethoxysilane, p-aminophenyltrimethoxysilane, N-phenylaminopropyltrimethoxysilane N-phenylaminopropyltriethoxysilane, n-butylaminopropyltrimethoxysilane, n-butylaminopropyltriethoxysilane, 3-(N-allylamino)propyltrimethoxysilane, (N,N-diethyl-3-aminopropyl)trimethoxysilane, and (N,N-diethyl-3-aminopropyl) triethoxysilane; and
(b) the trimethylsilyl group capping agent is selected from the group consisting of 1,1,1,3,3,3-hexamethyldisilazane (HMDS), trimethylmethoxysilane, phenyldimethylmethoxysilane and octyldimethylmethoxysilane.

7. The protocell of claim 5 or 6, wherein the protocell's antibiotic release profile is dependent upon the extent of silica framework condensation during nanoparticle aerosol-assisted evaporation-induced self-assembly.

8. The protocell of any of claims 1-7, wherein the mesoporous silica nanoparticle is conjugated to the lipid bi- or multilayer by cholesterol-containing tether molecules which are covalently linked to the mesoporous silica nanoparticle, either directly or through a PEG group.

9. The protocell of any of claims 1-8, wherein the lipid bi- or multilayer comprises:
(a) at least one zwitterionic lipid selected from the group consisting of 1, 2-dioleoyl-sn-glycero-3-phosphocholine (DOPC), 1, 2-dioleoyl-sn-glycero-3-phosphoethanolamine (DOPE) and 1,2-distearoyl-sn-glycero-3-phosphocholine (DSPC);
(b) optionally, one or more additional electrically charged or neutral lipids selected from the group consisting of 1,2-dipalmitoyl-sn-glycero-3-phosphocholine (DPPC), dioleylglycero triethyleneglycyl iminodiacetic acid (DOIDA), distearylglycerotriethyleneglycyl iminodiacetic acid (DSIDA), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof;
(c) one or more endo/lyso/phagosomolytic peptides that are incorporated into the lipid bi- or multilayer; and
(d) optionally, one or more nucleic acid sequences that are loaded into the nanoparticle and that are complementary to a gene sequence expressed by the one or more bacteria.

10. The protocell of any of claims 1-7, wherein:
(a) the host cells are selected from the group consisting of innate immune cells, alveolar type II epithelial cells, hepatocytes, macrophages and dendritic cells;
(b) the lipid bi- or multilayer is comprised of 1,2-dioleoyl-5-glycero-3-phosphocholine (DOPC), 1, 2-dioleoyl-sn-glycero-3-phosphoethanolamine (DOPE) and mannosylated cholesterol;
(c) the targeting ligands are selected from the group consisting of RGD (Arg-Gly-Asp), Fcγ (synthesized with a C-terminal cysteine residue), a single-chain antibody fragment against DEC-205, human complement C3, monophosphoryl lipid A, ephrin B2, GE11, and SP94;
(d) the endo/lyso/phagosomolytic peptide is H5WYG (synthesized with a C-terminal cysteine residue);
(e) optionally, the lipid bi- or multilayer incorporates one or more additional components selected from the group consisting of the self signal CD47, a polymerizable lipid and an acid-labile cross-linker;
(f) the nucleic acid sequences are selected from the group consisting of small interfering RNA, small hairpin RNA, microRNA, peptide nucleic acid, and spherical nucleic acids (SNAs), the nucleic acid sequences being complementary to one or more of a β-lactamase gene, a single-gene determinant of antibiotic resistance, a gene that contributes to virulence and a RNA polymerase or gyrase; and
(g) the one or more bacteria are selected from the group consisting of F. tularensis, B. pseudomallei, B. mallei, Coxiella burnetti, Yersinia pestis, Bacillus anthracia, Staphylococcus aureus, Klebsiella pneumoniae, and P. aeruginosa.

11. The protocell of any of claims 1-10, wherein the one or more antibiotics are selected from the group consisting of Gentamicin, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Cephalothin, Cephalexin, Cefaclor, Cefamandole, Cefoxitin, Cefprozil, Cefuroxime, Cefixime, Cefdinir, Cefditoren, Cefoperazone Cefotaxime, Cefpodoxime, Ceftazadime, Ceftibuten, Ceftizoxime Ceftriaxone, Cefepime, Ceftaroline fosamil, Ceftobiprole, Teicoplanin, Vancomycin, Telavancin, Daptomycin, Oritavancin, WAP-8294A, Azithromycin, Clarithromycin, Dirithromycin, Erythromycin, Roxithromycin, Telithromycin, Spiramycin, Clindamycin, Lincomycin, Aztreonam, Furazolidone, Nitrofurantoin, Oxazolidonones, Linezolid, Posizolid, Radezolid, Torezolid, Amoxicillin, Ampicillin, Azlocillin, Carbenicillin, Cloxacillin Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Demeclocycline, Doxycycline, Vibramycin Minocycline, Tigecycline, Oxytetracycline, Tetracycline, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

12. The protocell of any of claims 1-8 and 11, wherein the PEGylated lipid bilayer comprises about 25% to about 70% by weight of 1,2-dioleoyl-5-glycero-3-phosphocholine (DOPC), about 5% to about 15% by weight of 1,2-dioleoyl-sn-glycero-3-phosphoethanolamine (DOPE), about 20% to about 40% by weight of cholesterol, and about 5% to about 20% by weight of PEG.

13. The protocell of any of claims 1-8 and 11, wherein the PEGylated lipid bilayer comprises to about 25% to about 70% by weight of 1,2-dioleoyl-5-glycero-3-phosphocholine (DOPC), about 5% to about 15% by weight of 1,2-dioleoyl-sn-glycero-3-phosphoethanolamine (DOPE), about 20% to about 40% by weight of cholesterol, and about 5% to about 20% by weight of PEG.

14. The protocell of claim 9, wherein:
(a) the target host cell is THP-1;
(b) the receptor is Fcγ from human IgG;
(c) the F. tularensis is subspecies holarctica live vaccine strain (LVS);
(d) the protocells exhibit burst release kinetics upon administration;
(e) the endo/lyso/phagosomolytic peptide is H5WYG; and
(f) the nanoparticles comprise about 1% to about 5% by weight of levofloxacin;
and wherein the cytotoxicity of the protocell exceeds that of free levofloxacin and levofloxacin-loaded 1,2-distearoyl-sn-glycero-3-phosphocholine (DSPC) liposomes.

15. The protocell of claim 10, wherein:
(a) the target host cell is THP-1;
(b) the receptor is Fcγ from human IgG;
(c) the F. tularensis is subspecies holarctica live vaccine strain (LVS);
(d) the protocells exhibit burst release kinetics upon administration;
(e) the endo/lyso/phagosomolytic peptide is H5WYG; and
(f) the nanoparticles comprise about 1% to about 5% by weight of levofloxacin;
and wherein the cytotoxicity of the protocell exceeds that of free levofloxacin and levofloxacin-loaded 1,2-distearoyl-sn-glycero-3-phosphocholine (DSPC) liposomes.

16. The protocell of any of claims 1-15, wherein the internal surface area of said nanoparticle is greater than about 750 m²/g and the core pore wall size is less than about 3 nm.

17. The protocell of claim 9, wherein the nucleic acid sequences are peptide nucleic acids (PNAs) and spherical nucleic acids (SNAs) which penetrate Gram-negative and positive bacteria and which are complementary to one or more β-lactamase genes.

18. The protocell of claim 10, wherein the nucleic acid sequences are peptide nucleic acids (PNAs) and spherical nucleic acids (SNAs) which penetrate Gram-negative and positive bacteria and which are complementary to one or more β-lactamase genes.

19. The protocell of claim 10, wherein H5WYG is conjugated to diacylphosphatidylethanolamine (PE) moieties on the surface of the lipid bi- or multilayer by an amine-to-sulfhydryl crosslinker with a PEG spacer, and wherein the targeting ligand is RGD (Arg-Gly-Asp), which is bound to the surface of the lipid bi- or multilayer by an acid labile crosslinker.

20. The protocell of claim 19, wherein the lipid bilayer is PEGylated with between about 5% by weight to about 15% by weight of PEG-2000.

21. A pharmaceutical composition comprising a plurality of protocells of any of claims 1-20 and, optionally, one or more pharmaceutically-acceptable excipients.

22. The pharmaceutical composition of claim 21, wherein the composition is orally administered and the protocells are enterically coated.

23. A method of treating a subject who suffers from one or more bacterial infections, the method comprising administering to the subject a pharmaceutically-effective amount of protocells according to any one of claims 1-20.

24. The method of treatment of claim 23, wherein the subject is infected by one or more biological warfare agents selected from the group consisting of Bacillus anthracis (anthrax), Burkholderia mallei (glanders), Burkholderia pseudomallei (melioidosis), Clostridium botulinum toxin (botulism), Francisella tularensis (tularemia), Vibrio cholerae (cholera) and Yersinia pestis (plague).

25. The method of treatment of claim 23 or 24, wherein the protocells comprise or are co-administered with one or more antibiotics selected from the group consisting of rifampicin, oxacillin, ampicillin, b-lactam antibiotics, rifamycin group antibiotics, ciprofloxacin, erythromycin, macrolides, methicillin, metronidazole, ofloxacin, penicillin, streptomycin, tetracycline and vancomycin.

26. The method of treatment of claim 23, wherein the subject is infected by one or more bacteria selected from the group consisting of Escherichia, Salmonella, Shigella, Citrobacter, Edwardsiella, Enterobacter, Hafnia, Klebsiella, Morganella, Proteus, Providencia, Serratia, and Yersinia, Pseudomonas, Burkholderia, Stenotrophomonas, Shewanella, Sphingomonas, Comamonas, Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Treponema, Borrelia, Leptospiraceae, Campylobacter, Helicobacter, Spirillum, Streptobacillus, Bacteroides, Fusobacterium, Prevotella, Porphyromonas, Acinetobacter, A. baumanii, Listeria monocytogenes, Staphylococcus aureus, Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium tuberculosis, Corynebacterium diphteriae, Mycoplasma pneumoniae, and Actinomyces.

27. The method of treatment of claim 23, wherein the subject suffers from respiratory tularemia and the pharmaceutically-effective amount of protocells are orally administered to the subject.

28. Use of a protocell according to any of claims 1-20 in the manufacture of a medicament for the treatment of a bacterial infection in a patient or subject.

29. Use according to claim 28 wherein said bacterial infection is one or more infections selected from the group consisting of Escherichia, Salmonella, Shigella, Citrobacter, Edwardsiella, Enterobacter, Hafnia, Klebsiella, Morganella, Proteus, Providencia, Serratia, and Yersinia, Pseudomonas, Burkholderia, Stenotrophomonas, Shewanella, Sphingomonas, Comamonas, Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Treponema, Borrelia, Leptospiraceae, Campylobacter, Helicobacter, Spirillum, Streptobacillus, Bacteroides, Fusobacterium, Prevotella, Porphyromonas, Acinetobacter, A. baumanii, Listeria monocytogenes, Staphylococcus aureus, Enterococcus faecalis, Enterococcus faecium, Streptococcus pneumoniae, Streptococcus pyogenes, Streptococcus mutans, Streptococcus equi, Clostridium difficile, Clostridium botulinum, Clostridium tetani, Clostridium perfringens, Bacillus anthracis, Bacillus cereus, Propionibacterium acnes, Mycobacterium avium, Mycobacterium tuberculosis, Corynebacterium diphteriae, Mycoplasma pneumoniae, and Actinomyces.
